(19) United States
(12) Patent Application Publication
(10) Pub. No.: US 2025/0280853 A1
(43) Pub. Date: Sep. 11, 2025

(54) HYDROLYSIS OF SEED PROTEIN CONCENTRATE IN SUBCRITICAL WATER MEDIA, PRESSURIZED FLUID MEDIA AND ELECTROLYSIS OR COMBINED TECHNOLOGIES WITH ADDITION OF CITRUS PECTIN AND CITRIC ACID

(71) Applicant: The Governors of the University of Alberta, Edmonton (CA)

(72) Inventors: Hung Vo, Edmonton (AB); Marleny D.A. Saldaña, Edmonton (CA)

(73) Assignee: The Governors of the University of Alberta, Edmonton, AB (CA)

(21) Appl. No.: 18/863,787

(22) PCT Filed: May 10, 2023
(86) PCT No.: PCT/CA2023/050640

§ 371 Date: Nov. 7, 2024

Related U.S. Application Data
(60) Provisional application No. 63/340,088, filed on May 10, 2022.
(60) Provisional application No. 63/347,196, filed on May 31, 2022.

(51) Int. Cl. A23J 3/14; A23J 1/14; A23J 3/32; C07K 1/12; C07K 14/415

(52) U.S. Cl. CPC A23J 3/14; A23J 1/146; A23J 3/32; C07K 1/12; C07K 14/415

(57) ABSTRACT

A method of producing peptides from seed protein includes mixing a seed protein concentrate with a catalyst to prepare a mixture, dissolving the mixture in a buffer to prepare a suspension, and hydrolysing the suspension in a subcritical water medium in a high pressure system that includes a temperature controller, a batch stirred reactor, and a heating mantle. Hydrolysing the suspension includes loading the suspension into the batch stirred reactor, purging the reactor with a purge gas while stirring the suspension therein for a purge time, pressurizing the reactor with a pressurizing gas to a reaction pressure, heating the reactor to a reaction temperature, maintaining the reaction pressure and the reaction temperature within the reactor for a reaction time, cooling the reactor to a post-reaction temperature after the reaction time has elapsed and depressurizing the reactor, and centrifuging a hydrolysate resulting from the hydrolysing.

### RELATED APPLICATIONS

This application claims the priority benefit of U.S. Provisional Application Ser. No. 63/340,088, filed on May 10, 2022 and U.S. Provisional Application Ser. No. 63/347,196, filed on May 31, 2022, the contents of which are hereby incorporated by reference.

### FIELD OF THE INVENTION

The present invention relates to methods of producing small molecular weight peptides or bioactive compounds from pea protein concentrate using subcritical water hydrolysis, pressurized fluid media, electrolysis or novel two-step sequential hydrolysis using electrolysis followed by subcritical water process. More particularly, it relates to the use of a green compound such as citrus pectin to enhance the degree of hydrolysis of pea protein to obtain high value products like peptides and oligosaccharides as well as protein nanofibrils. These products can be used in films, gels, encapsulants, and emulsions.

### BACKGROUND OF THE INVENTION

The consumption of protein worldwide is witnessing a major shift from animal-based to plant-based sources. It is not only because plant protein is a high quality, affordable, and sustainable food source; but it also offers multiple potential health benefits, such as reduce the risk of cardiovascular diseases, hypertension, diabetes, LDL cholesterol level, and balance blood sugar level (Boukid et al., 2021; Klost et al., 2020). Pea, Pisum sativum, is one of the pulse crops that can provide a considerable amount of protein derived from the pea seeds. Canada is the world largest pea producer, its production yield was 4.2 million tons in 2019, followed by Russia, China, and the USA (FAOSTAT. 2021; Canadian Grain Commission. 2020). The protein content in pea seeds can range from 23.3% to 31.7% depending on the genotypes, environment, and soil factors (Reinkensmeier et al., 2015). The commercial pea protein products commonly are pea flour, pea protein concentrate, and pea protein isolate listed from the lowest to the highest protein content. The production of pea flour simply involves milling of pea seeds without any further separation. Pea flour can be directly fractionated further by air classification into two fractions, the coarse fraction is rich in starch, and the fine fraction is rich in protein (i.e., protein concentrate with 50-80% protein) (Reinkensmeier et al., 2015). The pea protein isolate (>80% protein) production requires more complicated purification steps. The most common purification method is alkaline isoelectric precipitation; however, this method can alter the native structure of protein due to the drying step (Pelgrom et al., 2015). Additionally, the large insoluble aggregates can also be formed in the final product (Chao & Aluko, 2018).

The majority portion of pea protein is globulin which accounts for 70-80%, and a minor portion of albumin which accounts for 10-20% of total protein (Acquah et al., 2020). Albumin (2S, 5-80 kDa) is a water-soluble protein, which contains more essential amino acids than globulin, such as tryptophan, lysine, threonine, and methionine (Boukid et al., 2021). Globulin is a salt soluble globular protein which is made up of three different fractions with different sedimentation coefficients and molecular masses. Legumin (11S, ˜360 kDa) is a hexameric protein whose subunits consist of a-acidic chain and β-basic chain linked together by disulfide bonds. Vicilin (7S, ˜180 kDa) is a trimer which is held together by hydrophobic interaction and lastly convicilin (7S, ˜290±40 kDa) is tetrameric fraction (Bogahawaththa et al., 2019; Klost & Drusch, 2019).

Recently, pea protein has drawn the attention of the scientific community due to its functional properties and its derived bioactive peptides. Several studies have demonstrated that pea protein hydrolysates possess antioxidant activities as well as health promoting properties. Li & Aluko. (2010) used Alcalase enzyme to hydrolyze the pea protein. The peptides fraction that had the most positive charge showed the strongest angiotensin converting enzyme (ACE) and renin inhibitory activities. In addition, the more hydrophilic the peptides, the stronger inhibitory activities against ACE and renin. There were three dipeptides identified to have moderate to strong ACE and renin inhibitory activities: IR (isoleucine-arginine), KF (lysine-phenylalanine), and EF (glutamic acid-phenylalanine). The authors were able to determine and characterize the health promoting effects of pea peptides, but the degree of hydrolysis (DH) as well as molecular mass of the peptides were not yet determined. Hence, Barbana & Boye. (2010) conducted research to investigate the changes in molecular weight (MW), degree of hydrolysis (DH), and ACE inhibitory activities of pea protein hydrolysates generated by enzymatic digestion. The MW range of the native protein was between 30 and 670 kDa, the MW distribution got smaller after enzymatic treatment, which was from 0.244 to 44 kDa. More than 50% of which had MWs below 4 kDa. Regarding the degree of hydrolysis, Alcalase and Flavourzyme (AF) combination could create the hydrolysate with the highest DH (78.58%) compared to other hydrolysates generated by other two enzymes: papain (31.18%) and gastrointestinal (31.08%). The antihypertensive effect of pea hydrolysate was eventually tested in humans (Li et al., 2011). The participants were orally administered pea protein hydrolysates daily for 3 weeks. They showed that with 3 g/day could result in a maximum 10 mmHg reduction in systolic blood pressure compared to the placebo group.

On the other hand, the antioxidant activities of pea protein have also been improved by hydrolysis mainly due to the formation of the peptides and amino acids. Pea protein hydrolysates were generated by thermolysin digestion in the study of Pownall et al. (2010), the scavenging activities against DPPH free radicals and metal ions were enhanced. After protein hydrolysis, the total content of hydrophobic amino acids (alanine, valine, isoleucine, leucine, etc.) increased leading to the improvement of DPPH scavenging activity accordingly. However, the ability to neutralize superoxide ions, hydroxyl radicals, and hydrogen peroxide did not significantly increase after protein depolymerization. Ding et al. (2020) reported the same results after hydrolyzing pea protein with alkaline protease, the DPPH radical scavenging rate of the hydrolysate fraction (M_{w}<1 kDa) was 37.9%. Contrasting with the previous study, the scavenging ability of pea protein hydrolysate against hydroxyl radical was reported to be 27.8%. Ding et al. (2020) identified three main peptides essentially contributing to the antiradical activities of the hydrolysates: YSSPIHIW (Tyr-Ser-Ser-Pro-Ile-His-Ile-Trp) (SEQ ID NO. 1), ADLYNPR (Ala-Asp-Leu-Tyr-Asn-Pro-Arg) (SEQ ID NO. 2), and HYDSEAILF (His-Tyr-Asp-Ser-Glu-Ala-Ile-Leu-Phe) (SEQ ID NO. 3).

Besides the enzymatic hydrolysis, pea protein hydrolysate can also be generated by heat- and pressure-pretreatment which was reported in the study of Chao et al. (2013) and Girgih et al. (2015). Both studies applied high pressure (200-600 MPa/24° C./5 min) or high temperature (100° C./30 min) prior to enzymatic treatment with Alcalase (1-4%). With the pretreatment of high pressure or temperature, lower concentration of Alcalase needed to generate low MW peptides. Though, at high pressures, 400-600 MPa, the protein became resistant to the proteolytic effect due to the extreme shielding effect resulting from protein-protein interactions. Moreover, the improvement of antioxidant activities was as well observed: superoxide and hydroxyl, DPPH, and oxygen radical scavenging. Probably under high pressure, 400 MPa, the peptide bonds were more exposed to Alcalase hydrolysis, resulting in the formation of bioactive peptides which led to the enhancement of free radical scavenging.

Enzymatic digestion is a well-known approach to hydrolyze protein; however, the method bears several disadvantages such as high-cost enzymes, time consuming, complex protocol, numerous buffer systems, and strictly controlled parameters. In addition, acid or base hydrolysis is also commonly used to breakdown protein, similarly, this method could generate environmentally harmful waste, toxic compounds (e.g., lysinoalanine), difficult in controlling peptides size, destroy and racemize the amino acids (Espinoza et al., 2012; Marcet et al., 2016; Sereewatthanawut et al., 2008). Therefore, subcritical water (sCW) has been employed as a green technology to hydrolyze protein or protein-rich biomass. Not only because sCW is environmentally friendly but it also has unique characteristics that could favor depolymerization reaction such as low dielectric constant, high ionic species, and high hydronium and hydroxide ion concentration (Simsek Kus, 2012; Zhao & Saldaña, 2019).

TABLE 1
Hydrolysis of protein-rich biomasses in subcritical water.

| | Hydrolysis | | |
|---|---|---|---|
| Material | conditions | Remarks | Reference |
| Deoiled rice | Temp: 100-220° C. | The highest protein (219 | Sereewatthanawut et |
| bran | Pressure: 1-39.7 | mg/g) and amino acids (8 | al. (2008) |
| | bar | mg/g) yields were obtained | |
| | Time: 5-30 min | at 200° C./30 min. | |
| | Material:water: | Hydrolysis time did not | |
| | 1:5 (w/v) | affect the protein and amino | |
| | | acids yields. | |
| | | Alkali hydrolysate had less | |
| | | protein and amino acids than | |
| | | sCW. | |
| | | Hydrolysate at 200° C./30 | |
| | | min showed the highest | |
| | | antioxidant activity and was | |
| | | the favorable medium for | |
| | | yeast growth. | |
| Rice bran | Temp: 100-360° C. | The highest total soluble | Pourali et al. |
| | Pressure: not | nitrogen was reached at | (2009) |
| | reported | 280° C. | |
| | Time: 5 min | Lysine and glutamic acid | |
| | Material:water: | had the highest contents | |
| | 3:18 (w/v) | among amino acids and | |
| | | peaked at 127° C. then | |
| | | decreased upon further | |
| | | heating; being degraded to | |
| | | carboxylic acids and gaseous | |
| | | products. | |
| | | Sucrose was detected with | |
| | | the highest concentration at | |
| | | 140° C. then degraded to | |
| | | fructose and glucose. | |
| | | Organic acids: formic, | |
| | | acetic, citric, glycolic, and | |
| | | levulinic acids were the | |
| | | degradation products of | |
| | | amino acids; which had the | |
| | | highest content at >190° C. | |
| Rice bran | Temp: 150° C.- | Protein aggregates reduced; | Sunphorka et al. |
| protein | 250° C. | polypeptides and amino | (2012) |
| | Pressure: 4.7-39.7 | acids yield increased after 5 | |
| | bar | min of heating. | |
| | Time: 5-60 min | Maximum amount of | |
| | Material:water: | polypeptides obtained at | |
| | 0.167:100, and | 250° C. for 20 min. | |
| | 0.416:100 (w/v) | Amino acid content | |
| | | increased with increasing | |
| | | temperature up to 250° C. | |
| | | without an observable | |
| | | maximum. | |
| Red algae | Temp: 120-230° C. | The highest hydrolysis | Park et al. |
| | Pressure: 30 bar | efficiency of 73.62% | (2019) |
| | Time: 30-50 min | reported at 210° C. | |
| | Material:water | Maximum DPPH (16.63 | |
| | 1:20 (w/v) | mg/g), ABTS (19.45 mg/g), | |
| | | and total phenolic content | |
| | | (14.29 mg/g) obtained at | |
| | | 210° C. | |
| | | Increasing temperature | |
| | | resulted in lower amino acids | |
| | | contents, with the highest | |
| | | content obtained at 120° C. | |
| | | (6273 mg/100 g). | |
| Soybean protein | Temp: 40-160° C. | Highest isoflavones yield | Benjamin et al. |
| isolate | Time: 4-14 min | (61%) obtained at 114° C., 2 | (2017) |
| (83% protein) | Pressure: 103 bar | min with a ratio of | |
| | Material:water: | solid/liquid of 1%. | |
| | 1:100, 4:100, and | Conversion of malonyl to | |
| | 7:100 (w/w) | glucosides forms occured | |
| | | at >60° C., glucosides to | |
| | | aglycones at >160° C. | |
| | | Temperatures did not | |
| | | significantly affect | |
| | | isoflavones recovery but | |
| | | could alter the structure of its | |
| | | derivatives. | |
| | | Highest amount of malonyl | |
| | | (23%) and glucosides (42%) | |
| | | obtained at 80° C. and 160° C., | |
| | | respectively. | |
| Soy protein | Temp: 90, 120° C. | Hydrodynamic diameter of | Wang et al. |
| isolate (85.79% | Time: 20 min | protein increased with | (2019) |
| protein) | Pressure: 0.7/1.9 | increasing temperature and | |
| | bar | protein concentration. | |
| | Material:water: | Partial hydrolysis occurred | |
| | 1:100, 2:100, and | in sCW condition. | |
| | 4:100 (w/v) | Partial unfolding led to the | |
| | | exposure of the molecule's | |
| | | interior containing | |
| | | hydrophobic fragments. | |
| | | Less exposed hydrophobic | |
| | | sites at 120° C. than 90° C., | |
| | | suggesting formation of | |
| | | aggregates. | |
| | | Heat treated proteins had | |
| | | more flexible confirmation, | |
| | | hence higher surface activity | |
| | | at air/water or oil/water | |
| | | interface. | |
| Soybean meal | Temp: 200-220° C. | Protein content increased | Watchararuji et al. |
| | Time: 10-30 min | with increasing temperature | (2008) |
| | Pressure: 1-39.7 | and time, maximum content | |
| | bar | of 165.72 mg/g at 210° C. for | |
| | Material:water 1:5 | 30 min. | |
| | and 2:5 (w/v) | Further heating at 220° C. | |
| | | could decrease protein | |
| | | yields. | |
| | | The highest amino acids | |
| | | yield (18.62 mg/g) was | |
| | | obtained at 210° C./30 min. | |
| | | At 220° C., reaction time did | |
| | | not greatly affect the amino | |
| | | acids content. | |
| Microalgae | Temp: 240-320° C. | Only arginine presented in | Garcia-Moscoso et al. |
| | Time: 6-12 s | significant amount after | (2015) |
| | Pressure: 207 bar | hydrolysis (127.8 mg/g) at | |
| | Material:water | 320° C./6 s. | |
| | 1:10 (w/v) | The highest soluble | |
| | | peptides (674.7 mg/g) | |
| | | obtained at 320° C./9 s. | |
| | | Gallic acid and Maillard | |
| | | reaction products could form | |
| | | in the hydrolysates. | |
| | | Phenol in the hydrolysates | |
| | | was 200-1000 times less than | |
| | | conventional process (300- | |
| | | 350° C./1 h) | |
| Okara extract | Temp: 170-260° C. | Highest protein content | Wiboonsirikul et al. |
| | Time: 2-120 min | (7 g/L) was obtained at | (2013) |
| | Pressure: not | 240° C., time had insignificant | |
| | reported | effect on protein content. | |
| | Material:water: | sCW could disrupt the | |
| | 2:100 (w/v) | strong aggregation of protein | |
| | | that resulted from | |
| | | hydrophobic interactions. | |
| | | Hydrolysate at 240° C./5 | |
| | | min had the highest | |
| | | antioxidant activity of 70%. | |
| | | Antioxidant activity was | |
| | | proportional to protein | |
| | | content. | |
| Brewer's spent | Temp: 105, 120, | BSG protein did not | Qin et al. |
| grains (BSG) | 135° C. | degrade to soluble peptides | (2018) |
| | Time: 15, 30, 60, | and amino acids under the | |
| | 120 min | investigated temperatures. | |
| | Pressure: not | Instead, more | |
| | reported | carbohydrates were soluble | |
| | Material:water: | in the hydrolysates. | |
| | 2.5, 4, 5, 6.67:100 | | |
| | (w/v). | | |
| Soy meals | Enzyme pre- | Protein yield, 59.3%, was | Lu et al. |
| | treatment | considerably higher than | (2016) |
| | Protease M | conventional methods. | |
| | Enzyme:substrate | Large protein aggregates | |
| | 4:100 (w/w). | were disrupted and | |
| | Temp: 50° C. | eventually improved | |
| | Time: 10-120 min | solubility. | |
| | sCW extraction | sCW treated soy protein | |
| | Temp: 120° C. | had higher neutral and | |
| | Time: 20 min | hydrophobic amino acids | |
| | Pressure: not | than the native one. | |
| | reported. | Longer hydrolysis time, | |
| | Material:water | higher content of | |
| | 1:10 (w/v). | hydrophobic amino acids. | |
| | | Plenty of aggregates were | |
| | | found, which were bigger | |
| | | than the native protein. | |
| | | Red-shifted in fluorescence | |
| | | spectrum indicated a more | |
| | | polar environment. | |
| | | Reduction in fluorescence | |
| | | intensity showed the partial | |
| | | unfolding of protein. | |
| Flaxseed meal | Temp: 130, 160, | Protein yield reached a | Ho et al. |
| | 190° C. | maximum of 225 mg/g meal | (2007) |
| | Time: 180-420 | at 160° C./400 min and pH 9. | |
| | min | Polar protein is more | |
| | Pressure: 340 bar | soluble at lower temperature, | |
| | Material:water | less polar protein is more | |
| | 1:90, 1:150, 1:210 | soluble at higher | |
| | (w/v) | temperature. | |

Even though sCW has been extensively used to hydrolyze protein from various sources, it has been known to have quite low specificity hydrolysis. Powell et al. (2016) concluded that cleavage could happen elsewhere in the protein molecules besides the breakdown of peptide bonds, possibly in the amino acids side chain. Previously, Rogalinski et al. (2005) noticed the low selectivity of sCW in bovine serum albumin hydrolysis. While various types of protein have been hydrolyzed or isolated using sCW technology (Table 1), there is no study investigating the hydrolysis reaction of pea protein concentrate in sCW media.

Peptides derived from pea protein have demonstrated its antioxidant activities as well as health promoting properties. The most common method to generate peptides is enzymatic digestion of pea protein. However, such method is costly due to the high enzymes' price, especially in the industrial scale process that require large amounts of enzymes. Furthermore, the hydrolysis duration can take up to a few hours to be completed. In addition, the hydrolysis protocol may involve different buffer systems and different temperatures, which make it complicated for large scale production. For all the above reasons, it is necessary in the art for the improvements in the treatment of pea protein for peptides or bioactives production.

Therefore, there exists a need to (1) evaluate the effect of reaction parameters (temperatures, times, and mixing ratios) on the protein degree of hydrolysis using one processing method or a two-step process, including electrolysis followed by subcritical water processing, (2) elucidate the interactions of pectin and citric acid with pea protein in sCW conditions, and (3) determine the antioxidant activities of the hydrolysate towards DPPH free radical scavenging.

### SUMMARY OF THE INVENTION

The present invention provides a method of producing peptides from seed protein that includes mixing a seed protein concentrate with a catalyst to prepare a mixture, dissolving the mixture in a buffer to prepare a suspension, and hydrolysing the suspension in a subcritical water medium in a high pressure system that includes a temperature controller, a batch stirred reactor, and a heating mantle. Additionally, hydrolysing the suspension includes loading the suspension into the batch stirred reactor, purging the reactor with a purge gas while stirring the suspension therein for a purge time, pressurizing the reactor with a pressurizing gas to a reaction pressure, heating the reactor to a reaction temperature, maintaining the reaction pressure and the reaction temperature within the reactor for a reaction time, cooling the reactor to a post-reaction temperature after the reaction time has elapsed and depressurizing the reactor, and centrifuging a hydrolysate resulting from the hydrolysing.

### BRIEF DESCRIPTION OF THE DRAWINGS

The subject matter that is regarded as the invention is particularly pointed out and distinctly claimed in the claims at the conclusion of the specification. The foregoing and other objects, features, and advantages of the invention are apparent from the following detailed description taken in conjunction with the accompanying drawings in which:

FIG. 1A is a graph showing the effect of reaction times at 180° C. on the degree of hydrolysis of pea protein and pectin suspensions;

FIG. 1B is a graph showing the effect of temperatures for 10 min on the degree of hydrolysis of pea protein and pectin suspensions;

FIG. 1C is a graph showing mass ratios for 180° C./10 min on the degree of hydrolysis of pea protein and pectin suspensions;

FIG. 2 shows a schematic representation of the protein hydrolysis reaction;

FIG. 3 is a graph comparing one step process (electrolysis) vs two-step process (electrolysis followed by sCW);

FIG. 4. is a graph comparing one step process (ultrasound) vs two-step process (ultrasound followed by sCW);

FIG. 5A is a graph of the protein content of the hydrolysates under the effects of reaction time at 180° C.;

FIG. 5B is a graph of the protein content of the hydrolysates under the effects of reaction temperature during 10 min;

FIG. 5C is a graph of the protein content of the hydrolysates under the effects of mass ratio of protein and pectin at 180° C./10 min;

FIG. 6. Emission fluorescence spectra of pea protein concentrate hydrolysates prepared by sCW hydrolysis with pectin (A, B);

FIG. 7A is a graph showing the distribution of peptide size in the hydrolysates of protein and pectin at 180° C./10 min;

FIG. 7B is a graph showing the distribution of peptide size in the hydrolysates of protein and pectin at 1:1 (w/w)/10 min;

FIG. 7C is a graph showing the distribution of peptide size in the hydrolysates of protein and pectin at 1:1 (w/w) and 180° C.;

FIG. 8A is a graph showing size exclusion chromatography of pea protein hydrolysates with citric acid at 10 min;

FIG. 8B is a graph showing size exclusion chromatography of pea protein hydrolysates with citric acid at 180° C.

FIG. 9A is a graph showing free radical scavenging activity of pea protein hydrolysates with pectin 1:1 (w/w) and citric acid 1:0.2 (w/w) at 10 min, in which the lower-case letters indicate the statistical difference between the samples with protein and pectin mixture and in which the; capital letters imply the statistical difference of samples with protein and citric acid mixture;

FIG. 9B is a graph showing free radical scavenging activity of pea protein hydrolysates with pectin 1:1 (w/w) and citric acid 1:0.2 (w/w) at 180° C., in which the lower-case letters indicate the statistical difference between the samples with protein and pectin mixture and in which the capital letters imply the statistical difference of samples with protein and citric acid mixture;

FIG. 9C is a graph showing free radical scavenging activity of pea protein hydrolysates with pectin 1:1 (w/w) and citric acid 1:0.2 (w/w) at and (C) 180° C./10 min, in which the lower-case letters indicate the statistical difference between the samples with protein and pectin mixture and in which the capital letters imply the statistical difference of samples with protein and citric acid mixture;

FIG. 10A is a graph showing emission fluorescence spectra of control (pea protein concentrate) and pea protein concentrate+pectin hydrolysate prepared by subcritical water hydrolysis at various temperatures/10 min;

FIG. 10B is a graph showing emission fluorescence spectra of control (pea protein concentrate) and pea protein concentrate+pectin hydrolysate prepared by subcritical water hydrolysis at 180° for various times;

FIG. 11A is a graph showing the effect of reaction times on the degree of hydrolysis of pea protein concentrate and its mixture with pectin;

FIG. 11B is a graph showing the effect of reaction temperatures on the degree of hydrolysis of pea protein concentrate and its mixture with pectin;

FIG. 12A is a graph showing the effect of reaction time on the peptide size of pea protein concentrate+pectin hydrolysates; and

FIG. 12B is a graph showing the effect of reaction temperature on the peptide size of pea protein concentrate+pectin hydrolysates.

### DESCRIPTION OF THE INVENTION

The present invention has utility as is a method to produce bioactives such as peptides from seed protein via hydrolysis in subcritical water with citrus pectin added as a catalyst. While the present invention is detailed herein with respect to pea protein as being exemplary of various seed sources, it is appreciated that other high protein seed crops are also readily processed according to the present invention. These other seed crops illustratively include soybean, peanut, common bean, mung bean, lupine, chickpea, fava bean, lentil, grass pea, and cowpea.

The present invention provides simple and innovated techniques for the treatment of pea protein concentrate to obtain small molecular weight bioactives such as peptides. The present invention employs water in subcritical conditions as the media with the addition of modifiers such as citrus pectin to hydrolyze better pea protein. By using subcritical water, the produced peptides have small molecular weight of 4-6 kDa. Advantageously, the present invention does not involve any hazardous chemicals. The resulting peptides from pea protein via hydrolysis in subcritical water with citrus pectin added as a catalyst are useful as vegan protein that has high digestibility; a functional beverage product; bodybuilding supplements; peptide nutraceuticals: antioxidant peptides, antihypertensive peptides, anti-inflammatory peptides, antimicrobial peptides; and peptide surfactants. The present invention can be used for other pulses such as canola, faba, lentil, etc.

Subcritical water is water in liquid state which has temperature from 100° C. (boiling temperature) to less than 374° C. (critical temperature). The hot water is pressurized in such a way that still remains liquid. Under high temperature and pressure, many of the anomalous properties of water are observed, such as: low dielectric constant, low viscosity, low density, and high dissociation constant. All of which can affect the solvating power as well as the reactivity of subcritical water. The viscosity of water reduces substantially at subcritical temperature which can enhance mass transfer rates. Hence, protein hydrolysis reactions can favorably be induced in subcritical water medium because of its superior transport properties. Moreover, because of having relatively high dissociation constant, subcritical water can induce the ionic reactions. The present invention provides the methods of generating up to 64.76% degree of hydrolysis of the protein hydrolysates.

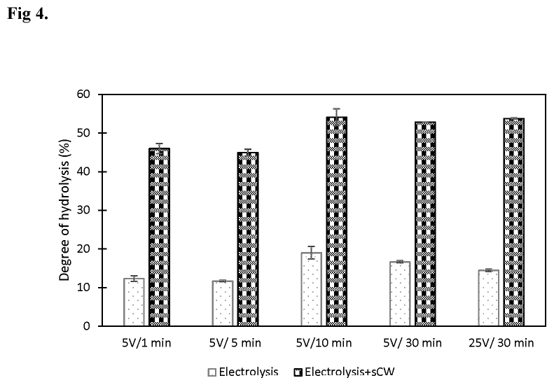

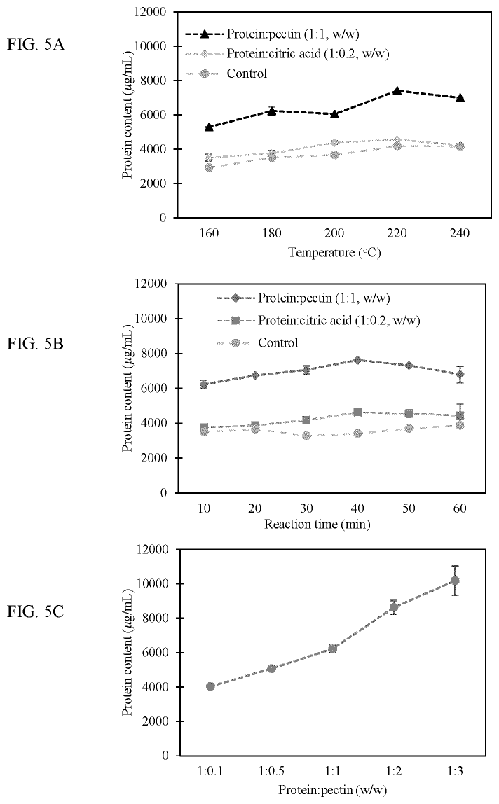

According to embodiments, a suspension is prepared by firstly mixing pea protein concentrate with citrus pectin at mass ratio of 1:1 (w/w). The mixture is then dissolved in 100 mL of 0.2M phosphate buffer at pH 8. After that, the suspension is stirred overnight under constant speed at 4° C. to hydrate the protein. The suspension of pea protein concentrate and citrus pectin is hydrolysed using subcritical water technology. The hydrolysis is conducted using a high pressure system which includes: a temperature controller, 600 mL batch stirred reactor and a 780W heating mantle. The suspensions are loaded into the 600 mL reactor then purged with N₂ gas for 12 min under constant stirring in order to avoid any undesirable oxidation reactions. After purging, the reactor is then pressurized with N₂ gas to a certain extent such that after reaching the desired temperature, a pressure value of 50 bar could be achieved. The reaction is performed at different temperatures (160, 180, 200, 220, and 240° C.) and times (10, 20, 30, 40, 50, and 60 min) under constant pressure of 50 bar. The reactor is immediately cooled down to 40° C. and depressurized after the reaction time elapsed. The resulting hydrolysate is centrifuged at 8,000 g/20 min to collect the supernatant. which is then stored at −18° C. until further characterizations. According to some inventive embodiments, the method additionally includes using ultrasound as a pretreatment step, or electrolysis as a pre-treatment step, the effects of which are shown in FIGS. 4 and 5.

It has been discovered that the selectivity of sCW is increased by adding 90% CO₂ to the media, as a result, the amino acid yield is improved by 4 times higher than sCW alone. It is further observed that a similar phenomenon occurs with pea hull fiber hydrolysis. It is additionally surprisingly observed that citric and malic acid enhances the selectivity of sCW towards cleavage of glycosidic bonds. Specifically, the content of gluco-oligosaccharides (2-6 DP) is approximately 5-fold higher compared to the media with solely water.

According to the present invention, pea protein concentrate, for the first time, is hydrolyzed by sCW with citrus pectin and citric acid as the additives. Pectin is able to assist with the generation of hydrolysates that have a high degree of hydrolysis. The highest DH is found to be 67.31% at 180° C./10 min, protein: pectin 1:3 (w/w). The hydrolysates with citric acid have significantly lower DH, the highest value is 34.66% at 240° C./10 min, protein: citric acid 1:0.2 (w/w). The effect of temperature is more pronounced than reaction time in terms of influencing the degree of hydrolysis. With increasing DH, the protein content of the hydrolysates accordingly increases. It is due, at least in part, to the breakdown of protein molecules into smaller peptides which have the MW ranging from 6.5-1.6 kDa. Among them, the most prominent peptide is 4.1 kDa that elutes at ˜32 min. All the protein hydrolysates experience the unfolding and the exposure of hydrophobic clusters which is indicated by the fluorescence intensity and the Amax values. The amino acid profiles show that hydrophilic amino acids str dominant in the hydrolysates, which account for at least 50% of total amino acids. The DPPH scavenging activity of hydrolysates with citric acid are appreciably higher than the ones with pectin. This indicates that pectin and citric acid assist the hydrolysis of pea protein concentrate in different manners. Overall, sCW is a green technology that has great potential to produce small MW peptides from pea protein concentrate, moreover, the specificity of sCW can be tailored to generate high DH, low antioxidant activity or low DH, high antioxidant activity hydrolysates by adding either citrus pectin or citric acid to the media for protein hydrolysis.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

### Degree of Hydrolysis

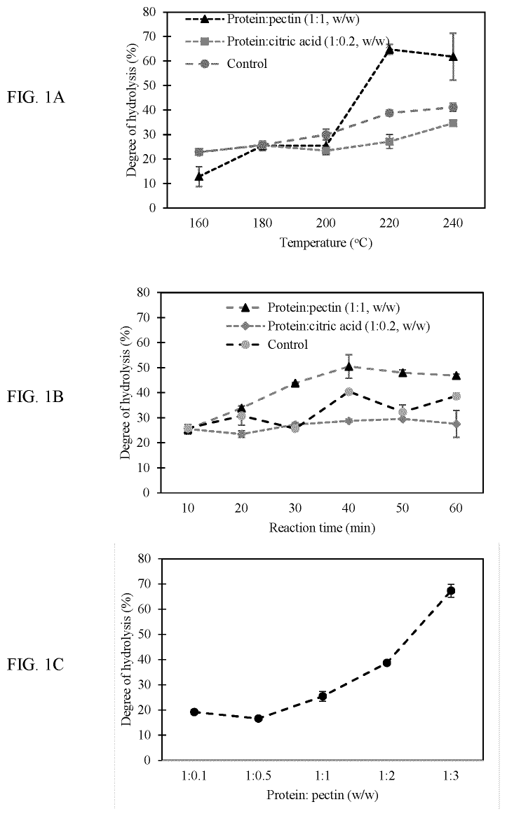

The degree of hydrolysis (DH) is indicated by the amount of free amino groups in the hydrolysates. The higher DH demonstrates the protein is cleaved into free amino groups at higher extent. With increasing time, the DH accordingly increases and reaches the maximum value of 50.5% at 40 min, as shown in FIG. 1A, afterwards, the DH remains unchanged even with longer times of 50 and 60 min. Similar results are observed in the study of Sereewatthanawut et al. (2008) after hydrolyzing rice bran using subcritical water. They report that the protein as well as amino acid yields did not increase with the increase of reaction time. In fact, the yields remained constant after 5 min until the end of the experiment at 30 min. Espinoza et al. (2012) noticed that the reaction time had no significant effect on the DH of whey protein hydrolysates. After 10 min of hydrolysis at 250° C., the DH values remained unchanged up to 60 min. The phenomenon could be attributed to the dissociation constant (Kw) of water, which is dependent on temperature and pressure of the media (Martinez-Monteagudo & Saldaña, 2014) i.e., the concentration of H₃O⁺ and OH⁻ ions do not change under constant pressure and temperature conditions. For that reason, the DH could not further increase after the proteins were hydrolyzed by the available ionic species in the sCW medium. Regarding the reaction time from 10 to 40 min, the DH is significantly enhanced due to better interaction between ionic species and protein.

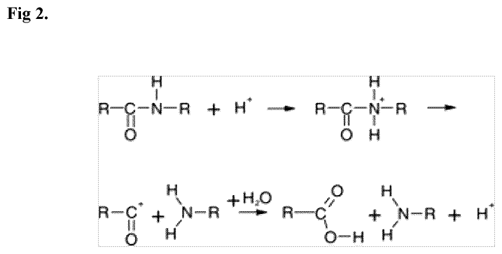

FIG. 1B shows the effect of temperature on the protein DH. When the temperature increasing from 160° C. to 200° C., the DH only increases slightly. However, the DH is considerably improved and peaks at 220° C. with the highest value of 64.8%. As mentioned earlier, the Kw value of water increases with temperature, Kw value at 220° C. is approximately 700 times higher than the one at ambient conditions (Saldaña et al., 2015; Sereewatthanawut et al., 2008). Consequently, the protein undergoes a more severe hydrolysis due to higher concentration of ionic species which could potentially break the peptide bonds in the protein; eventually increasing the amount of free amino groups in the hydrolysates. Ramachandraiah et al. (2017) reported hydrolysis reaction of soybean powder using sCW where they observed that when the temperature increased from 150° C. to 190° C., the content of free amino groups appreciably increased from 28 mg/L to 89 mg/L. The yield of amino acids in the study conducted by Pinkowska & Oliveros. (2014) had the similar changes that it was considerably increased at 220° C. then remained constant up to 260° C. and eventually decreased upon higher temperatures (260° C. to 300° C.). The possible mechanism of peptide bond cleavage by H₃O⁺ is shown in FIG. 2

Interestingly, the mixing ratio of pea protein and pectin has a noticeable effect on the DH. Particularly, with the increasing concentration of pectin, the DH is significantly enhanced. The maximum DH value is 67.3% at ratio pea protein: pectin 1:3 (w/w) (FIG. 1C). Pectin is a heterogeneous polysaccharide which has D-galacturonic acid (GalA) as the predominant monomer (60%) which are bonded together by glycosidic linkages (Valdivieso Ramirez et al., 2021). Rogalinski et al. (2008) suggested that the stability of peptide bonds is considerably higher than glycosidic bonds. Therefore, pectic substance could be broken down into its monomers, D-galacturonic acids and other hexuronic acids before the protein is hydrolyzed. The released acidic sugars from pectin then act as a catalyst for the hydrolysis reaction of protein. Earlier, Valdivieso Ramirez et al. (2021) reported that with the addition of 0.2% citric acid to sCW, the yield of gluco-oligosaccharides (2-6 DP) was 5 times higher than the one with sCW only. Rogalinski et al. (2005) used sCW media with 90% CO₂ and reported that the yield of amino acids from bovine serum albumin hydrolysis increased by 4 times compared to the one without CO₂ addition. For that reason, at higher concentrations of pectin, there would be more acidic sugars released into the reaction media which further hydrolyzed the protein, resulting in a higher degree of hydrolysis.

The pea protein hydrolysis reaction is also conducted in sCW media modified by citric acid. It is noticeable that the reaction time does not exert a significant effect on the DH of the hydrolysates (FIG. 1A), which ranges slightly from 23.5% to 27.6% during hydrolysis. On the other hand, the processing temperatures have a more pronounced effect on the DH (FIG. 1B), specifically, at 240° C., the DH value reaches the maximum of 34.6% meanwhile it is only 22.9% at 160° C. Compared to the media with pectin, the aqueous citric acid media has a considerably less proteolytic effect, resulting in a significantly lower DH. The pH of the suspensions is the primary reason attributed to the cleavage of pea protein. In the media with pectin, the pH value is maintained at 8 which is far above the isoelectric point of pea protein (˜4.3). The protein molecules are then negatively charged due to the alkaline environment used. Therefore, the possibility to combine with hydrogen ions is appreciably increased compared to the positively charged ones (Steinhardt & Fugitt. 1942). Eventually, the proton donated by pectic acidic sugars could integrate and break down the peptide bonds leading to the escalation of free amino groups in the samples with pectin added. On the contrary, the pH of aqueous citric acid solution was 2.6 which had the protein positively charged. Eventually reduced the hydrolysis potential of hydronium ions in the sCW media. Moreover, the proteolytic potential of the basic solution is proven to be more effective than acidic one, probably due to the recombination of peptides under acidic pH.

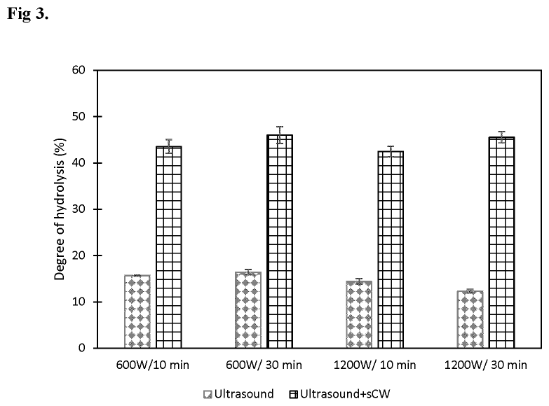

Electrolysis is known as an effective method to modify the structure of animal protein by breaking down the disulfide bridges (Cayot et al., 2002). Therefore, electrolysis was used as a pretreatment of pea protein concentrate hydrolysis followed by sCW. FIG. 3 shows the effect of electrical voltage and time on the DH of pea protein hydrolysates. At the conditions investigated, the voltage had no significant influence on the DH of pea protein. However, the electrolyzing time significantly enhanced the DH. The highest DH was 54.1% obtained at 5V/10 min followed by sCW hydrolysis at 1800C/40 min. Using longer or shorter time than 10 min, there was no effect of electrolyzing time on the hydrolysis of pea protein. Legume protein in general (Gorissen et al., 2018) and pea protein, in particular, contains relatively low amount of sulfur containing amino acids (see TABLE 2). Therefore, disulfide bonds may be present in a limited extent which do not require large energy nor long time to be hydrolyzed. Consequently, 5V for 10 min could be sufficient pretreatment to cleave —S—S— bonds in the protein. Overall, the use of a two-stage approach (electrolysis+sCW) was better than using only electrolysis.

Electrolysis is known as an effective method to modify the structure of animal protein by breaking down the disulfide bridges (Cayot et al., 2002). Therefore, electrolysis was used as a pretreatment of pea protein concentrate hydrolysis followed by sCW. FIG. 4 shows the effect of electrical voltage and time on the DH of pea protein hydrolysates. At the conditions investigated, the voltage had no significant influence on the DH of pea protein. However, the electrolyzing time significantly enhanced the DH. The highest DH was 54.1% obtained at 5V/10 min followed by sCW hydrolysis at 180° C./40 min. Using longer or shorter time than 10 min, there was no effect of electrolyzing time on the hydrolysis of pea protein. Legume protein in general (Gorissen et al., 2018) and pea protein, in particular, contains relatively low amount of sulfur containing amino acids (see Table 2). Therefore, disulfide bonds may be present in a limited extent which do not require large energy nor long time to be hydrolyzed. Consequently, 5V for 10 min could be sufficient pretreatment to cleave —S—S— bonds in the protein. Overall, the use of a two-stage approach (electrolysis+sCW) was better than using only electrolysis.

FIG. 5 shows the sequential process of ultrasound followed by sCW for the hydrolysis of pea protein concentrate. Even though the effect of time and sonication power had no significant effect on the DH of pea protein, with increasing time from 10 to 30 min, the DH improved from 43.5 to 46%. The power influenced the DH in an opposite way. At high power of 1200W, the DH was lower than the one obtained at 600W. Using the acoustic energy from the ultrasound, the interactions within protein such as Van de Waals forces, hydrogen bonds, and dipole attractions were disrupted (Li et al., 2016). Therefore, the use of ultrasound as a pre-treatment favored the hydrolysis with subcritical water, resulting in higher DH. The use of a two-stage approach (ultrasound+sCW) was better than using only ultrasound.

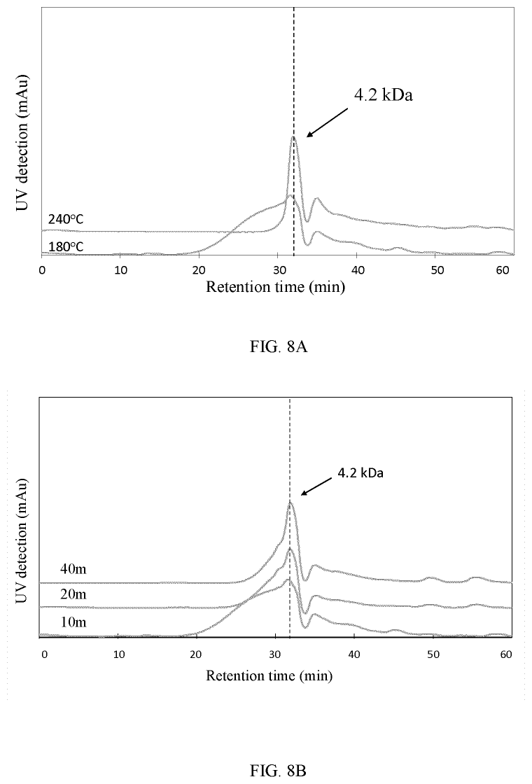

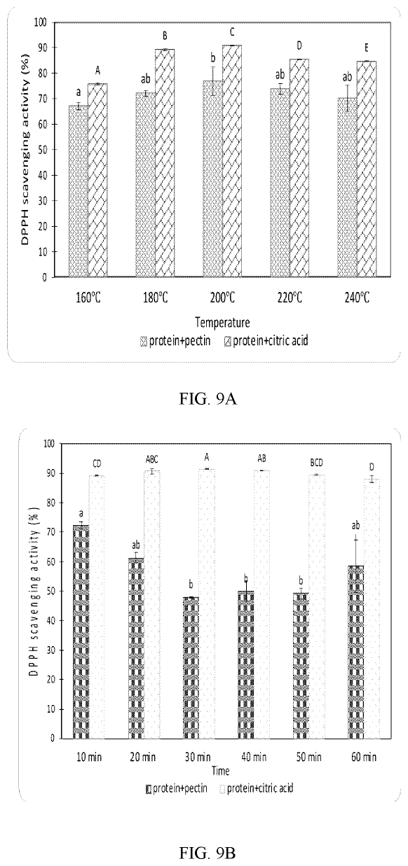

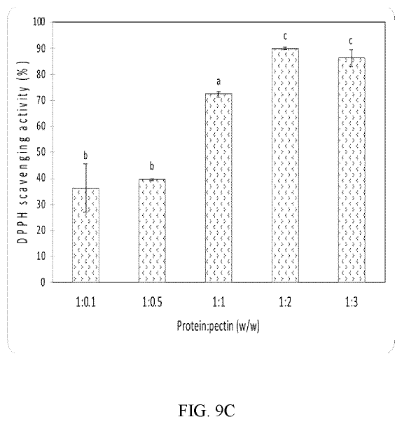

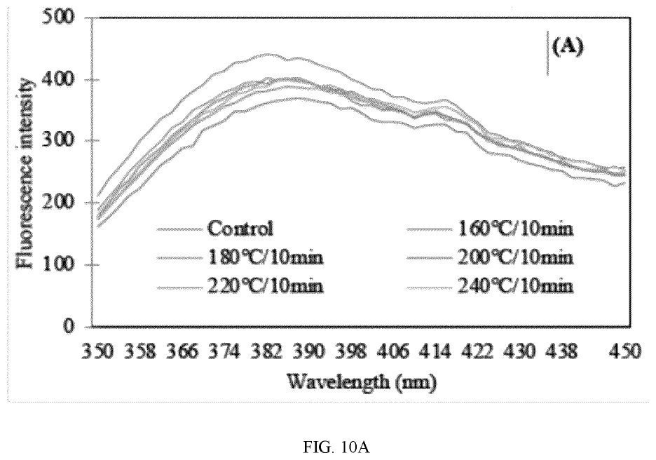

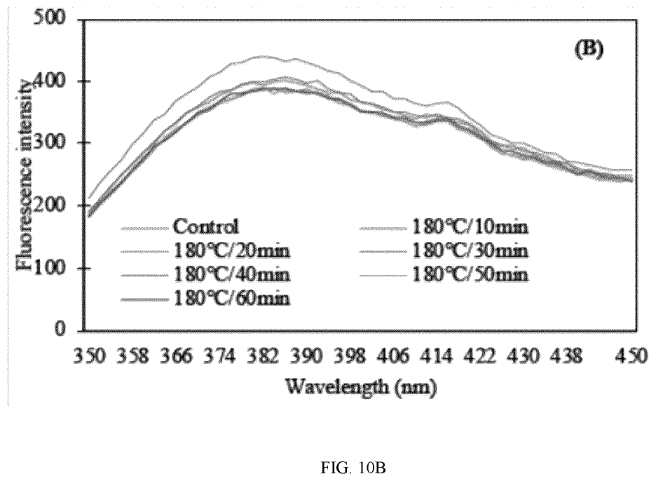

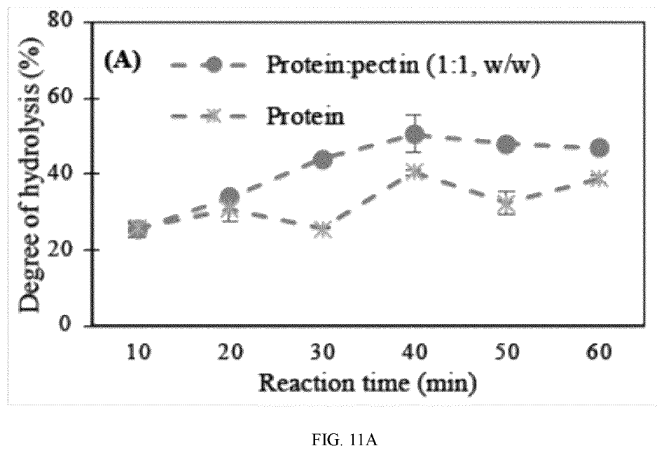

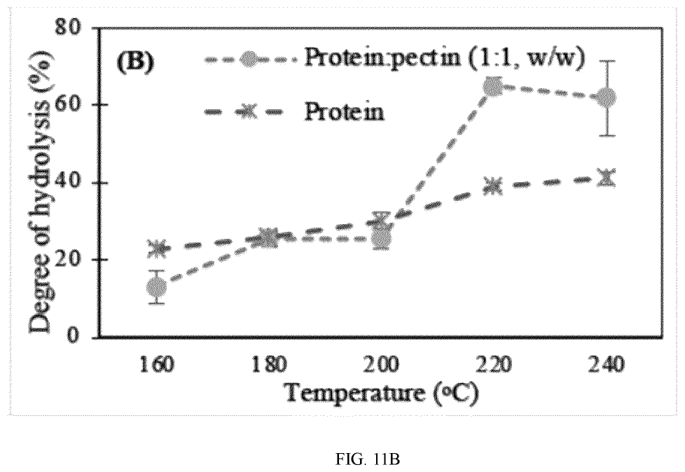

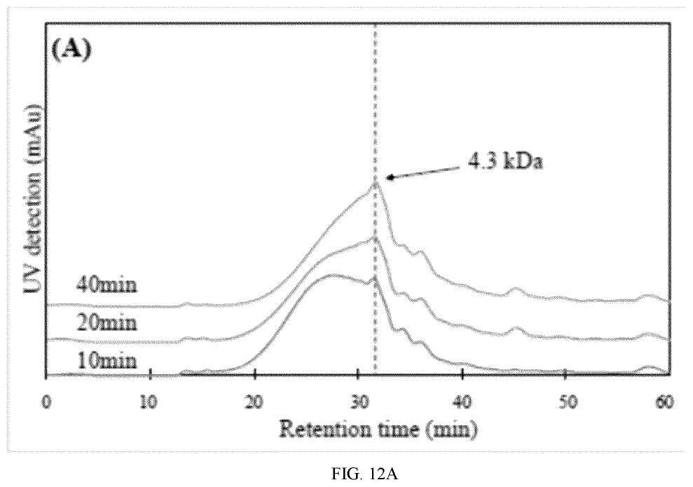

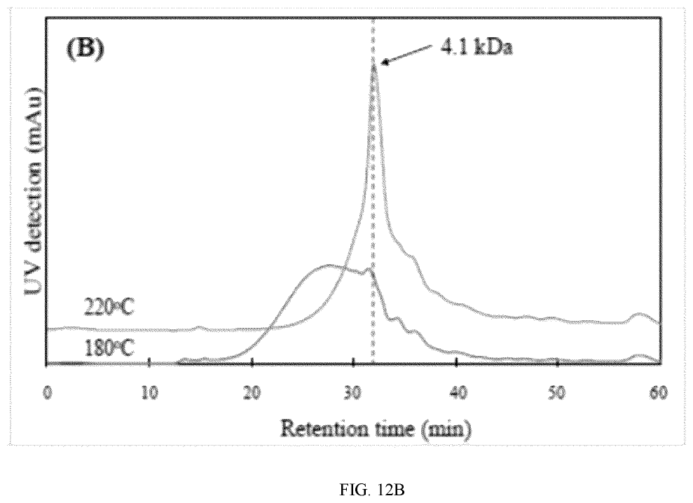

Comparing FIGS. 8 and 9, the use of electrolysis followed by sCW was more efficient than using ultrasound followed by sCW. FIG. 4 shows the sequential process of ultrasound followed by sCW for the hydrolysis of pea protein concentrate. Even though the effect of time and sonication power had no significant effect on the DH of pea protein, with increasing time from 10 to 30 min, the DH improved from 43.5 to 46%. The power influenced the DH in an opposite way. At high power of 1200W, the DH was lower than the one obtained at 600W. Using the acoustic energy from the ultrasound, the interactions within protein such as Van de Waals forces, hydrogen bonds, and dipole attractions were disrupted (Li et al., 2016). Therefore, the use of ultrasound as a pre-treatment favored the hydrolysis with subcritical water, resulting in higher DH. The use of a two-stage approach (ultrasound+sCW) was better than using only ultrasound.

Comparing FIGS. 3-4, the use of electrolysis followed by sCW was more efficient than using ultrasound followed by sCW.

### Protein Content of Hydrolysates

The protein content is determined by Lowry's method, which detects peptide bonds from its reaction with copper ions and Folin reagent. The resulting complex has a strong blue color which is detectable at 550 nm (Waterborg, 2009). The protein content steadily improves when the reaction time increases from 10 to 30 min; then reaches the maximum value of 7620.67 ug/mL at 40 min. With prolonged heating periods from 40 to 60 min, the protein content considerably reduces, Lu et al. (2016) reported the similar behavior of soy protein hydrolysis reaction, under the effect of extended heating time (>30 min), a significant decrease in protein yield is observed (FIG. 3A). Additionally, Sunphorka et al. (2012) noticed a similar behavior in rice bran protein hydrolysis, that the protein content increased up to 60 min of processing time. However, within the first period of the reaction (5 min), the proteins aggregates then assembles into solid precipitates. Afterwards, with the increasing reaction time, the particles disaggregates and releases the unfolded structure of protein, smaller soluble peptides, and amino acids. As a result, the protein content of the hydrolysates is then improved. The results agree with the findings in the study of Sereewatthanawut et al. (2008) who reported the protein content of soy protein hydrolysates gradually increased at longer holding time (from 10 to 30 min). The results are in agreement with the protein degree of hydrolysis where the DH value significantly changes at 10 to 40 min of reaction time. Under high temperature and pressure conditions, the structure of globular proteins could be unfolded (Chao et al., 2013; Lu et al., 2016). Therefore, more peptide bonds, which used to be buried inside, are exposed; hence, provided better interactions of the peptide bonds with the assay's reagents. Consequently, the protein content is increased.

On the other hand, the protein suspensions supplemented with citric acid have a quite limited increase in protein content, specifically the highest value was 4627.33 ug/mL obtained at 40 min, which is only 1.23 times higher than the lowest value at 10 min. The major reason, as mentioned above, could be due to the pH of the reaction medium. As citric acid supplemented, the pH value dropped to 2.6 which is below the isoelectric point of pea protein, hence, the protein molecules obtain the positive charges; eventually, reduce the hydrolytic ability of H₃O⁺ ions.

FIG. 3B shows the effect of temperatures on the protein content of the hydrolysates. With the increasing temperatures from 160° C. to 220° C., the protein contents are progressively increasing and peak at 220° C. (7400.67 ug/mL). Afterwards, it remains almost constant at 240° C. with the value of 6987.33 μg/mL. The results agree with findings in the studies of Ndlela et al. (2012) who reported that the protein contents of soybean protein hydrolysates were enhanced from 68% to 73% as temperatures increase from 150° C. to 234° C. Sunphorka et al. (2012) noticed a similar phenomenon for rice bran protein hydrolysis with increasing temperature, the protein content significantly increased. The dissociation of aggregated protein particles and unfolding of the protein structure could be likely leading to the increase in peptide bonds of the hydrolysates as explained previously. The hydrolysates with citric acid added have considerably lower protein content, the highest content is obtained at 220° C. with the value of 4564 μg/mL and increases upon further heating at a higher temperature of 240° C.

The protein contents of the hydrolysates are consistent with the degree of hydrolysis in FIG. 1B where the DH value also peaked at 220° C. However, there is no sudden increase observed in the protein content when the temperatures went from 200° C. to 220° C., which is unlike the result in FIG. 1B. The employed assays could be a reason attributed to the variation between two sets of data. The degree of hydrolysis is quantified by OPA assay which could detect the free amino groups in the hydrolysates. On the other hand, Lowry's method is used to determine protein content which would react with peptide bonds of the protein molecules. Therefore, it is obvious that the peptide bonds in the hydrolysates does not increase but instead, broken down after the treatment at 220° C. Increasing the amount of free amino groups in the hydrolysates which is indicated by FIG. 1B. To confirm the cleavage of peptide bonds, peptide size distribution is estimated using high performance size exclusion chromatography.

The mixing ratio of protein and pectin significantly influences the protein content of the hydrolysates (FIG. 3C). With increasing concentrations of pectin, there is more protein released in the hydrolysates. The content of protein greatly increases, approximately 2.5 times, from 4027.33 μg/mL to 10180.67 μg/mL when the ratio of protein and pectin increase from 1:0.1 to 1:3 (w/w). As discussed earlier, pectin can be broken down into hexuronic acids under sCW conditions; the more pectin, the higher the content of hexuronic acids in the hydrolysate. Therefore, pectic acids together with H₃O⁺ ions in the media can hydrolyze the protein in a higher extent i.e., they disrupt the inter- and intramolecular linkages: H-bonds, electrostatic and hydrophobic interactions, and disulfide bonds leading to the unfolding of its structure, eventually increasing the protein content of the hydrolysates (Zhang et al., 2018). Another reason that contributes to the increase in protein content is the pH of the media which can also markedly influence the conformational changes of protein molecules. At pH 8, which is far above the isoelectric point (˜4.3) of pea protein, the electrostatic repulsion and ion hydration are enhanced, therefore, the unfolding of protein is induced (Wen et al., 2019).

### Intrinsic Fluorescence Spectroscopy

Fluorescence spectroscopy is a sensitive method to monitor the conformational changes of protein tertiary structure. The fluorescence intensity of protein is largely attributed to the aromatic amino acid residues. FIGS. 5A and 5B show that sCW treatment could have modified the conformation of pea protein concentrate, which is indicated by the intensity of the emission spectra and the maximum emission wavelength (λₘₐₓ).

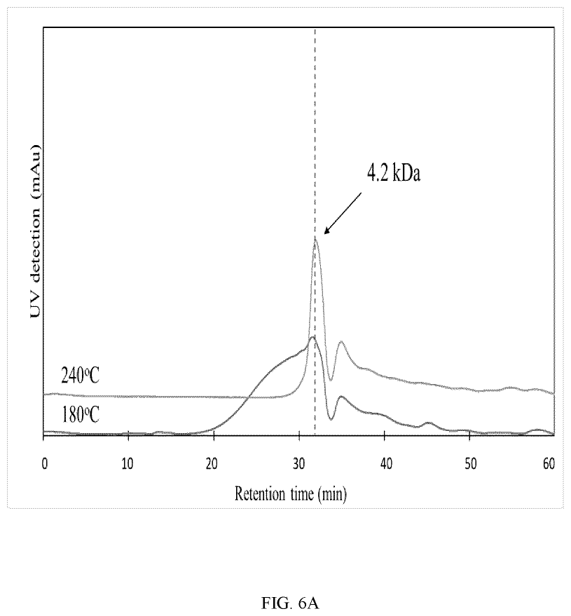

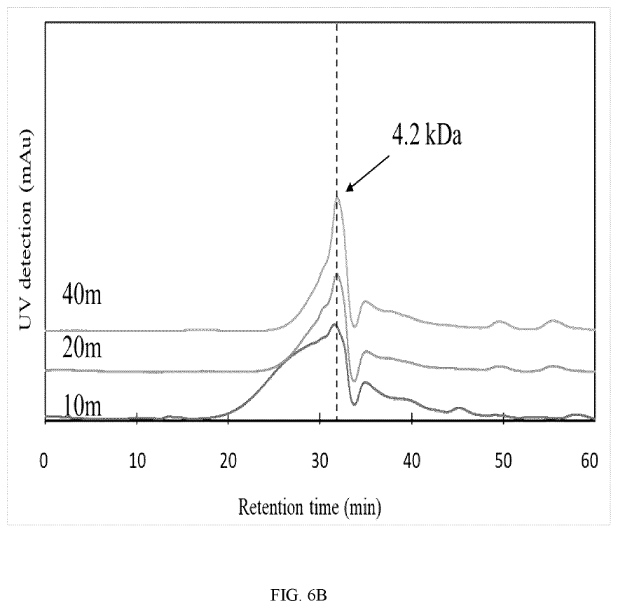

Compared to the untreated sample, all sCW-hydrolyzed protein with pectin have appreciably lower fluorescence intensity (FIGS. 6A and 6B). The decrease in fluorescence intensity could indicate the changes in the protein microenvironment which shifted to more polar. In other words, the unfolding of the protein occurs leading to the exposure of the interior hydrophobic clusters to the surface of the molecules. Lu et al. (2016) observed similar behavior when they hydrolyzed soy protein using sCW, reporting enhanced surface hydrophobicity. Chao et al. (2018) reported the reduction in fluorescence intensity of pea protein after 600 MPa treatment. They suggested that the protein could be severely denatured and unfolded to expose the structures that were previously buried in the interior. The maximum emission wavelength is another indicator of protein denaturation level (Schmid. 1989). Compared to the untreated sample which had λₘₐₓ at 382 nm, the sCW hydrolyzed proteins experienced a slight red-shifting in λₘₐₓ ranging from 384 nm to 388 nm. This is explained by the exposure of tryptophan residues to the aqueous environment. The interactions of aromatic amino acids with aqueous media enhance the fluorescence quenching of water therefore resulting in the lower fluorescence intensity and the red shift of λₘₐₓ. A similar observation is reported in the study of Chao et al. (2013) where the λₘₐₓ of pea protein slightly shifted from 372 nm to 375-378 nm after the high pressure pretreatment (200-600 MPa). The change of λₘₐₓ from short to long wavelengths is an indication of the extensive denaturation of protein and exposure of interior hydrophobic regions.

For the hydrolysis of pea protein concentrate with citric acid, the λₘₐₓ values of the hydrolyzed proteins are in the range of 382-386 nm and have slightly red-shifted compared to the untreated one (λₘₐₓ=382 nm) and the fluorescence intensity is also reduced (FIGS. 5C and 5D). Chao et al. (2018) observed that at pH 3, the intensity of fluorescence signal was considerably lower than the one at pH 5 and 7. They suggested that at pH 3, the protein was excessively denatured and exposed the tryptophan residues that were previously buried in the interior. The extensive interactions of tryptophan residues with the aqueous environment increases the fluorescence quenching of water, as a result, the fluorescence intensity was reduced. In this study, the pH of the citric acid and protein suspensions are 2.6, a similar phenomenon could have occurred to the pea protein. Another reason is that the protein-protein interactions are increased at pH 2.6 due to the isoelectric point of pea protein being 4.3. The lack of repulsive forces and the associated steric hindrance result in the reduced fluorescence intensity (Chao & Aluko, 2018).

The temperature and time treatment can reduce the fluorescence intensity and shift the λₘₐₓ of protein however, there is no linear relationship observed between the fluorescence intensity and sCW treatments. Chang et al. (2022) hydrolyzed egg white protein using sCW and reported that temperatures did not correlate to the fluorescence intensity of the protein and λₘₐₓ of the hydrolysates remained unchanged after sCW treatment.

### Peptide Size Distributions

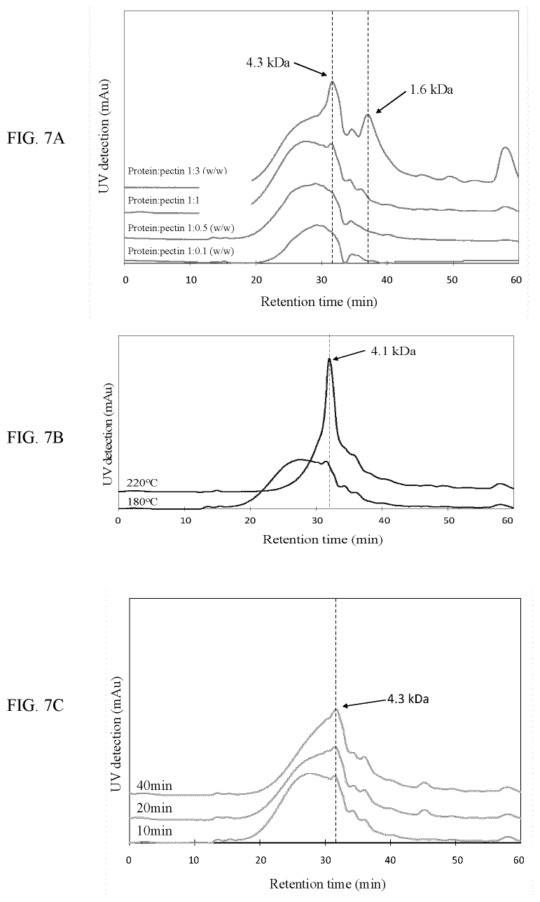

The changes in the molecular weight of peptide chains are monitored by size exclusion chromatography, depicted in FIGS. 7-8. At 40 min, the intensity of 4.3 kDa peak considerably increased. Also, the peak area of 4.3 kDa at 20 min and 40 min are 1.8 and 5.5 times higher than the one obtained at 10 min, respectively. This observation agreed with the degree of hydrolysis (FIG. 1A) where the highest DH is obtained at 40 min, which is likely due to the high content of peptides in the hydrolysate. Compared to reaction time, temperature has the most significant effect in reducing the peptides molecular mass. With increasing temperature, the better hydrolysis and the smaller peptides are therefore produced. The peptides found at 180° C. had molecular masses between 4.5-4.9 kDa with relatively low intensity from 53-55 mAu. However, at higher temperature of 220° C., the peaks eluted at 30 and 31 min disappear, instead, there is a prominent peak detected at 32 min with a considerably higher intensity of 153 mAu. The molecular mass of the identified peptides is 4.1 kDa. As shown in the chromatograms, the intensity of peptides at 220° C. is approximately 3 times higher than the one at 180° C. The area of 4.1 kDa peak at 220° C. is 6.5 times higher than the one at 180° C., suggesting that the proteolytic activity of the media is enhanced at higher temperature. The result well-explains the increment of the degree of hydrolysis at 220° C. in FIG. 1B which is due to the higher content of peptides in the media.

The mixing ratios of pectin and protein also show a noticeable effect on the hydrolysis of protein. At the ratios of 1:0.1 and 1:0.5 protein: pectin (w/w), there are no considerable changes in the peptides profile of the hydrolysates, the major peak is eluted at approximately 29 min corresponding to the MW of 6.3-6.5 kDa. However, at the ratios of 1:1 and 1:3 protein: pectin (w/w), the smaller peptides gradually appear with the retention times from 30-37 min which correlated to the MW of 4.9-1.6 kDa, respectively. Wang et al. (2019) observed a similar result when they hydrolyzed soy protein with sCW. At 120° C., the MW of the peptides was decreased to less than 75 kDa and the percentage of aggregated protein particles was reduced. For that reason, they suggested that under sCW treatment the disulfide bonds were broken, limiting the formation of aggregated particles.

Regarding the hydrolysis of pea protein with citric acid (FIG. 8), the molecular weight distributions have a relatively similar elution pattern. The prominent peak which has MW of 4.2 kDa, is observed in every chromatogram and eluted at ˜32 min. The peak's intensity gradually increases and reaches a maximum of 72 mAu at 240° C./10 min. The reaction time does not greatly affect the intensity of the ˜32 min peak, there is only a slight increase in its intensity from 49 to 56 mAu at the reaction times of 20 and 40 min, respectively.

### Amino Acid Profile

The amino acid profiles of pea protein and pectin hydrolysates are shown in Table 2. The dominant amino acid is to be Glu which content is found up to 43.12 g/100 g protein at 240° C. This agrees with the data reported by Gorissen et al. (2018) where glutamic acid is the most abundant amino acid in pea protein. Most of the essential amino acids are degraded at temperatures higher than 180° C., especially Thr, Arg, and Ser, which are undetectable at 240° C. The result is similar to the findings of Hao et al. (2019) where Thr is not found in the hydrolysates of abalone viscera extract at 230° C. On the other hand, the hydrophobic amino acids have increasing trends at higher sCW temperatures, particularly, hydrophobic amino acids contents are 33.08, 35.93, and 42.08 g/100 g at 160° C., 180° C., and 220° C., respectively. Among the amino acids obtained, Met/Val (5.36 g/100 g protein), Phe (8.85 g/100 g protein), and Ala (12.74 g/100 g protein) have the highest contents at 240° C. This result agrees with the findings in the study of Ziero et al. (2022) that the content of hydrophobic amino acids (Ile, Phe, Trp) are higher at elevated temperatures, suggesting that the hydrophobic characteristics of the hydrolysates are enhanced by the high temperatures of sCW which relates to the exposure of hydrophobic amino acids in the interior of protein molecules to the surface; as it is previously proposed by the fluorescence intensity in FIGS. 5A and 5B. The dielectric constant of sCW decreases at increasing temperature which favors the dissolution of non-polar molecules. The hydrophobic amino acids normally possess non-polar side chains which solubility is considerably enhanced at high temperature of sCW.

TABLE 2
Total amino acid composition of the protein and pectin hydrolysates.

| Amino | 180° C./10 min | | | | | | | Protein:pectin 1:1 | | |
|---|---|---|---|---|---|---|---|---|---|---|
| acids | Protein:pectin | Protein:pectin | Protein:pectin | Protein:pectin 1:1 (w/w), 10 | | | | (w/w), 180° C. | | |
| (g/100 g | 1:0.1 | 1:1 | 1:3 | min | | | | 10 | 40 | 60 |
| protein) | (w/w) | (w/w) | (w/w) | 160° C. | 180° C. | 220° C. | 240° C. | min | min | min |
| Asp | 15.67 | 15.7 | 15.05 | 15.43 | 15.7 | 5.27 | 1.7 | 15.7 | 14.03 | 12.73 |
| Glu | 24.65 | 26.24 | 24.36 | 23.39 | 26.24 | 31.62 | 43.12 | 26.24 | 26.95 | 27.72 |
| Ser | 2.33 | 2.56 | 4.94 | 5 | 2.56 | 0.44 | 0 | 2.56 | 1.93 | 2 |
| His | 3.09 | 3.38 | 3.03 | 2.9 | 3.38 | 3.15 | 2.5 | 3.38 | 3.25 | 3.08 |
| Gly | 7.79 | 7.5 | 6.83 | 6.37 | 7.5 | 9.73 | 9.26 | 7.5 | 7.38 | 8.46 |
| Thr | 1.03 | 1.34 | 3.84 | 3.55 | 1.34 | 0 | 0 | 1.34 | 1.18 | 1.07 |
| Arg | 3.7 | 1.32 | 1.06 | 2.37 | 1.32 | 0 | 0 | 1.32 | 0.99 | 0.69 |
| Ala | 6.91 | 7.79 | 6.93 | 6.41 | 7.79 | 10.39 | 12.74 | 7.79 | 8.14 | 8.33 |
| Tyr | 4.11 | 4.37 | 5.1 | 5.56 | 4.37 | 5.14 | 5.37 | 4.37 | 4.94 | 4.37 |
| Met/Val | 4.2 | 4 | 4.17 | 3.93 | 4 | 4.88 | 5.36 | 4 | 4.53 | 4.3 |
| Phe | 6.82 | 7.09 | 6.58 | 6.47 | 7.09 | 7.9 | 8.85 | 7.09 | 7.03 | 7.12 |
| Ile | 6.25 | 6.54 | 6.32 | 6.16 | 6.54 | 6.33 | 5.29 | 6.54 | 6.63 | 6.78 |
| Leu | 10.48 | 10.51 | 10.33 | 10.11 | 10.51 | 12.58 | 2.75 | 10.51 | 11.08 | 11.1 |
| Lys | 2.97 | 1.65 | 1.46 | 2.36 | 1.65 | 2.55 | 3.07 | 1.65 | 1.95 | 2.24 |
| Acidic^{a} | 40.32 | 41.94 | 39.41 | 38.82 | 41.94 | 36.89 | 44.82 | 41.94 | 40.98 | 40.45 |
| Basic^{b} | 9.76 | 6.35 | 5.55 | 7.63 | 6.35 | 5.7 | 5.57 | 6.35 | 6.19 | 6.01 |
| Uncharged | 15.26 | 15.77 | 20.71 | 20.48 | 15.77 | 15.31 | 14.63 | 15.77 | 15.43 | 15.9 |
| polar^{c} | | | | | | | | | | |
| Hydrophobic^{d} | 34.66 | 35.93 | 34.33 | 33.08 | 35.93 | 42.08 | 34.99 | 35.93 | 37.41 | 37.63 |
| Hydrophilic^{e} | 50.08 | 48.29 | 44.96 | 46.45 | 48.29 | 42.59 | 50.39 | 48.29 | 47.17 | 46.46 |

^{a}Asp, Glu.
^{b}His, Arg, Lys.
^{c}Ser, Gly, Thr, Tyr.
^{d}Ala, Met/Val, Phe, Ile, Leu.
^{e}Asp, Glu, His, Arg, Lys.

It is noteworthy that the contents of hydrophilic amino acids are the highest compared to other amino acid categories. In general, hydrophilic amino acids increase at increasing temperatures and reach the maximum value of 50.39 g/100 g protein at 240° C. The exposure of hydrophilic amino acids suggest the potential to chelate the ferrous ions of pea protein hydrolysates (Phongthai et al., 2018). The reaction times again do not considerably affect the changes in amino acids profiles of pea protein hydrolysates. There is a slight increase in hydrophobic amino acids content from 35.93 to 37.63 g/100 g protein at 10 and 60 min of reaction times, respectively. However, Sunphorka et al. (2012) contrastingly reported the amino acid contents of rice bran protein hydrolysates significantly increase with longer time from 10 to 60 min, the possible reason is due to the differences in temperature. The authors employed sCW at 250° C. which was much higher than this study, 180° C. Hence, under higher temperature, the amino acids contents increase.

Table 3 shows the amino acid profiles of pea protein and citric acid hydrolysates. Similarly, Glu is the most prominent amino acid in the hydrolysate with the maximum content of 40.05 g/100 g protein at 240° C. Most of the amino acids are unstable at high temperature, 240° C., except for the hydrophobic ones which content is improved with increasing temperature and peaked at 240° C. with 39.52 g/100 g that explains the unfolding of protein at high temperature, which is previously indicated by the intrinsic fluorescence intensity in FIGS. 6C and 6D. Most of the amino acid contents are not significantly influenced by the reaction times.

TABLE 3
Total amino acid composition of the protein and citric acid hydrolysates.

| Amino acids | | | | | | |
|---|---|---|---|---|---|---|
| (g/100 g | Protein:citric acid 1:0.2 (w/w), 10 min | | | Protein:citric acid 1:0.2 (w/w), 180° C. | | |
| protein) | 160° C. | 180° C. | 240° C. | 10 min | 20 min | 40 min |
| Asp | 13.97 | 11.95 | 1.56 | 11.95 | 10.3 | 7.55 |
| Glu | 21.56 | 23.77 | 40.05 | 23.77 | 23.59 | 26.84 |
| Ser | 5.66 | 5.63 | 1.29 | 5.63 | 5.59 | 5.46 |
| His | 3.09 | 3.38 | 3.06 | 3.38 | 2.91 | 3.29 |
| Gly | 5.61 | 6.08 | 8.21 | 6.08 | 5.91 | 6.47 |
| Thr | 4.35 | 4.19 | 0.98 | 4.19 | 4 | 4.21 |
| Arg | 9.88 | 7.98 | 1.95 | 7.98 | 7.97 | 6.39 |
| Ala | 5.47 | 5.97 | 10.58 | 5.97 | 6.14 | 6.86 |
| Tyr | 3.01 | 4.03 | 0 | 4.03 | 5.22 | 2.94 |
| Met/Val | 3.11 | 3.35 | 4.72 | 3.35 | 3.73 | 4.06 |
| Phe | 5.65 | 5.88 | 6.65 | 5.88 | 6.12 | 6.43 |
| Ile | 4.92 | 5.28 | 6.08 | 5.28 | 5.64 | 6.18 |
| Leu | 8.63 | 9.09 | 11.49 | 9.09 | 9.45 | 10.07 |
| Lys | 5.07 | 3.42 | 3.38 | 3.42 | 3.44 | 3.26 |
| Acidic^{a} | 35.53 | 35.72 | 41.61 | 35.72 | 33.89 | 34.39 |
| Basic^{b} | 18.04 | 14.78 | 8.39 | 14.78 | 14.32 | 12.94 |
| Uncharged | 18.63 | 19.93 | 10.48 | 19.93 | 20.72 | 19.08 |
| polar^{c} | | | | | | |
| Hydrophobic^{d} | 27.78 | 29.57 | 39.52 | 29.57 | 31.08 | 33.6 |
| Hydrophilic^{e} | 53.57 | 50.50 | 50.00 | 50.50 | 48.21 | 47.33 |

^{a}Asp, Glu.
^{b}His, Arg, Lys.
^{c}Ser, Gly, Thr, Tyr.
^{d}Ala, Met/Val, Phe, Ile, Leu.
^{e}Asp, Glu, His, Arg, Lys.

### DPPH Scavenging Activity of Pea Protein Hydrolysates

The antioxidant activity of pea protein hydrolysates is estimated by its ability to neutralize DPPH⁻ free radicals. Once DPPH encounters proton-donating species, its absorbance reduces thus reflect the scavenging ability of the species. The reaction time does not appreciably influence the antioxidant activity of pea protein hydrolysates with citric acid (FIG. 9A), however, all the hydrolysates possess relatively high antioxidant activity. At least 88% of DPPH-free radicals are scavenged. On the other hand, the protein hydrolysates with pectin are significantly affected by the reaction time, the highest antioxidant activity is 72.27% at 10 min, decreasing upon further heating.

Regarding the effect of temperature, the antioxidant activity of protein with both citric acid and pectin hydrolysates gradually increases to maximum values of 90.88%, and 76.95%, respectively, at 200° C., however, higher temperatures of 220 and 240° C. result in the reduction of antioxidant activity. This could be related to the peptide size and composition presented in the hydrolysates. At lower temperatures of 160-200° C., there are different peptides with various MW detected (FIGS. 7-8); it is likely that these peptides could have encountered DPPH radicals then transformed them to more stable products; hence, contributed to the antioxidant activity of the hydrolysates. At higher temperatures of 220 and 240° C., the degradation of high MW peptides is clearly observed (FIG. 7), there is only one dominant peptide with MW of ˜4.1 kDa detected which probably has lower antioxidant activity than the ones at 200° C. Consequently, the antioxidant activity of the hydrolysates at 220 and 240° C. is reduced. Another possible reason is about the content of aromatic amino acids that can donate hydrogen atoms from the hydroxyl groups to neutralize the free radicals (Phongthai et al., 2018). As indicated in Table 2, the contents of aromatic amino acids (Tyr, and Phe) considerably decrease from 9.91 to 6.65 g/100 g protein at 180 and 240° C., respectively; therefore, that might affect the antioxidant activity of the hydrolysates. Compared to citric acid hydrolysates, the protein and pectin hydrolysates show a lower scavenging activity against DPPH indicated in FIGS. 9A and 9B. This potentially demonstrates that pectin and citric acid catalyze pea protein hydrolysis reaction in different patterns, thus different peptides are formed, leading to the differences in antioxidant activity. It is noteworthy to mention that the antioxidant activity seems to be inversely proportional to the degree of hydrolysis of pea protein. The higher DH, the lower antioxidant activity. A similar observation was reported in the study of Klompong et al. (2007) that with increasing DH of yellow stripe trevally animal protein from 5 to 25%, the DPPH scavenging activity is reduced from approximately 97% to 78%, respectively.

Gomes & Kurozawa. (2020) investigated the antioxidant capacity of rice protein hydrolyzed by Alcalase and Flavourzyme. They reported that with increasing degree of hydrolysis, the DPPH scavenging activity increased as the smaller peptides are more effective to engage with DPPH radicals, consequently increased the antioxidant activity of the protein hydrolysates. However, the data reported in the present invention does not completely agree with the mentioned finding. The degree of hydrolysis at 200° C. and 220° C. are found to be 25.1% and 64.8% respectively; however, the antioxidant activity at 220° C. is significantly lower than the one at 200° C. It is noteworthy that Gomes & Kurozawa. (2020) only looked at the DH from 1 to 10 of rice protein. Meanwhile, in the present invention the hydrolysates are obtained with appreciably higher degree of hydrolysis, from 13% to 67%. Therefore, the antioxidant activity is not proportionally related to the degree of hydrolysis but depends on the peptide size and its composition.

The invention is further illustrated by way of Example.

### Example 1

### Materials and Methods

Pea protein concentrate (51% protein) is provided by AGT Food and Ingredients Inc. (Saskatoon, SK, Canada). Citrus pectin (30% degree of esterification) is supplied by CP Kelco (Atlanta, GA, USA). Chemicals involved in the sCW hydrolysis are citric acid (>99.5%, ACS grade) from Sigma Aldrich (Oakville, ON, Canada), potassium phosphate monobasic from ICN Biomedicals, Inc. (Aurora, OH, USA), sodium hydroxide (>95%) from Fisher Scientific (Ottawa, ON, Canada), water from the Milli-Q system (18.2 MΩ cm, Millipore, Billerica, MA, USA), and nitrogen gas (99.9% purity) from Praxair (Edmonton, AB, Canada).

For characterizations, all chemicals are in analytical grade. Sodium tetrahydroborate, sodium dodecyl sulfate, sodium carbonate, sodium potassium tartrate, copper (II) sulfate, β-mercaptoethanol, o-Phthaldialdehyde, Folin-Ciocalteu reagent, 2,2-diphenyl-1-picrylhydrazyl, L-Lysine and bovine serum albumin are obtained from Sigma Aldrich (Oakville, ON, Canada). Methanol (99.9%) is purchased from Fisher Scientific (Ottawa, ON, Canada). The calibration standards for peptide size exclusion chromatography were blue dextran (2000 kDa), bovine serum albumin (66.4 kDa), carbonic acid (29 kDa), cytochrome C (12.4 kDa), aprotin (6.5 kDa), and L-configurations standard amino acids for amino acid HPLC analysis are purchased from Sigma Aldrich (Oakville, ON, Canada).

### Subcritical Water Hydrolysis

The suspensions are prepared by firstly mixing pea protein concentrate with pectin at different mass ratios of 1:0.1, 1:0.5, 1:1, 1:2, and 1:3 (w/w), and pea protein concentrate with citric acid at ratio of 1:0.2 (w/w). The mixtures are then dissolved in 100 mL of 0.2M phosphate buffer at pH 8 using a Heidolph homogenizer (Heidolph Instruments GmbH & Co., Germany) at 3,200 rpm for 1 min. After that, the suspensions are stirred overnight under constant speed at 4° C. Lastly, subcritical water is employed to hydrolyze the suspensions according to the procedure described by Valdivieso Ramirez et al. (2021) with slight modifications.

The hydrolysis is conducted using a Parr 4590 system (Parr Instrument Company, Moline, IL, USA) with the optimized proportional, integral, and derivative parameters for the temperature controller are 21, 500, and 71, respectively. The system is also equipped with a 600 mL batch stirred reactor and a 780W heating mantle. The stirring speed is estimated around 660 rpm (1.5 knob position on the controller's panel). The suspensions are loaded into the 600 mL reactor then purged with N₂ gas for 12 min under constant stirring in order to avoid any undesirable oxidation reactions. After purging, the reactor is pressurized with N₂ gas to a certain extent such that after reaching the desired temperature, a pressure value of 50 bar is achieved. The reaction is performed at different temperatures (160, 180, 200, 220, and 240° C.) and times (10, 20, 30, 40, 50, and 60 min) under constant pressure of 50 bar. The reactor is immediately cooled down to 40° C. and depressurized after the reaction time elapsed. The resulting hydrolysates are centrifuged at 8,000 g/20 min to collect the supernatant, which is then stored at −18° C. until further characterizations.

### Characterizations of the Protein Hydrolysates

### Degree of Hydrolysis

The extent of protein hydrolysis is measured by spectrophotometry method based on the reaction of primary amine and OPA with the presence of β-mercaptoethanol to form an OPA adduct that could be detected at 340 nm (Church et al., 1983). The procedure is adopted from Mirzaei et al. (2015) with slight modifications. Briefly, OPA reagent is prepared by mixing 40 mg of OPA dissolved in 1 mL of methanol, 25 mL of 0.1M sodium tetrahydroborate, 2.5 mL of 20% SDS (w/v), and 100 μL of β-mercaptoethanol are later added. The final volume is adjusted to 50 mL using mili-Q water. The OPA reagent is freshly prepared daily. To perform the assay, 0.1 mL of the hydrolysate is mixed with 2 mL of OPA reagent and incubated for exactly 2 min at room temperature. Finally, the absorbance is read at 340 nm using a UV-Vis spectrophotometer. The concentration of free amino group is estimated by a calibration curve with L-Lysine as the standard (0-2 mM). The degree of hydrolysis (DH) is calculated using Equation 1.

$$DH ( \% ) = \frac{L - L o}{Lma x - Lo} \times 100 \qquad Equation 1$$

where, L is the concentration of free amino groups after hydrolysis, Lₒ is the concentration of free amino groups of the untreated sample, and Lₘₐₓ is the concentration of free amino groups obtained by conventional acid hydrolysis (6M HCl at 110° C./24h).

### Total Protein Content

The total protein content is reflected by the color intensity which is formed by two reactions: (i) reaction with alkaline copper, and (ii) reduction of Folin-Ciocalteu reagent. The procedure is adopted from Waterborg (2009) with minor modifications. Firstly, 1 mL of 4-fold-diluted hydrolysate is mixed with 1 mL of 2N NaOH solution then incubated at 100° C./10 min in a water bath. The solution is then cooled down to room temperature and mixed with 10 ml of complex-forming reagent (100 mL of 2% (w/v) sodium carbonate+1 mL of 1% (w/v) copper (II) sulfate+1 mL of 2% (w/v) sodium potassium tartrate). After 10 min at room temperature, 1 mL of Folin-Ciocalteu reagent is added to the solution. The mixture stands in the dark at room temperature for 35 min. The absorbance is read at 550 nm using the UV-Vis spectrophotometer. The protein content is calculated by a calibration curve with bovine serum albumin as the standard (0-2 mg/mL).

### Total Amino Acids Profile

The hydrolysate is treated with hydrochloric acid before determination of amino acids profile; by mixing 1 mL of the hydrolysate with 6 mL of with 6M HCl at 110° C./24h. The mixture is then mixed with 0.2 mL of internal standard which includes B-amino-n-butyric acid and ethanolamine at 25 μmol/mL; followed by centrifugation at 2500 rpm/15 min. The vials for HPLC injection included 50 μL of the supernatant, 50 μL of 4.29M NaOH, and 400 μL of mili-Q water. More NaOH solution is added to maintain pH of the solution at 9, which is crucial for derivatization. The mobile phase includes two eluents: (A) 1600 mL of 0.1M sodium acetate buffer pH 7.2, 180 mL methanol, 10 mL tetrahydrofolic acid, and 210 mL of mili-Q water; (B) 100% methanol. The elution gradient is: 0-1 min, isocratic 100% A; 5-25 min, isocratic 85% A and 15% B; 38-39 min, linear from 55% to 35% A and 45% to 65% B; 40-42.5 min, isocratic 100% B, and 43-48 min, isocratic 100% A. The separation is performed by Supelcosil LC-18 column (Sigma Aldrich, Oakville, ON, Canada), 150 mm×4.6 mm, 3 μm with the flow rate of 1.1 mL/min. The fluorescence intensity of the samples is measured at the wavelength of 340 nm (excitation) and 450 nm (emission). The calibration curves are prepared by three different concentrations of L-configuration amino acids (150, 300, and 600 nM).

### Peptide Molecular Weight Distribution

Size exclusion chromatography is used to evaluate the size distribution of peptides. The procedure is adopted from (Klost & Drusch, 2019) with minor modifications. The diluted hydrolysates are filtered and injected through a Superdex 75 Increase 10/300 GL (GE healthcare GmbH, Solingen, Germany) column with 0.1M phosphate buffer at pH 7 as the mobile phase. The UV detector is used at 280 nm. The estimation of peptide molecular weight distribution is carried out via calibration standards (Sigma Aldrich, Oakville, ON, Canada): blue dextran (2000 kDa), bovine serum albumin (66.4 kDa), carbonic acid (29 kDa), cytochrome C (12.4 kDa), and aprotin (6.5 kDa).

### Intrinsic Fluorescence Spectroscopy

The intrinsic fluorescence intensity of protein is mainly contributed by aromatic hydrophobic amino acids: tyrosine, phenylalanine, and tryptophan. The fluorescence intensity indicates the conformational changes of the protein molecules (Lakowicz. 2006). The procedure is previously described by Chao & Aluko. (2018) with minor modifications. Briefly, the hydrolysates are diluted with phosphate buffer pH 8 or aqueous citric acid until the protein concentration reached 20 μg/mL. The solutions are then centrifuged at 10,000 g/5 min to collect the supernatant. The fluorescence intensity of protein solutions are scanned with a spectrofluorometer (SpectraMax® M3, Molecular Devices, San Jose, CA, USA) with excitation wavelength of 295 nm and emission spectra from 350 nm to 450 nm with 2 nm increment.

### Scavenging of DPPH Free Radical

The scavenging ability of the hydrolysates is determined according to the method of Zhang et al. (2008). Briefly, 0.1 mM DPPH solution is prepared with absolute methanol. Then, 1 mL of 2-fold-diluted hydrolysates was mixed with 3 mL of DPPH solution then stands at room temperature for 35 min. After that, the solution is centrifuged at 7000 rpm/5 min to collect the supernatant. The absorbance of the supernatant is read at 514 nm using the UV-Vis spectrophotometer. The scavenging ability is expressed as the percentage of scavenging ability using Equation 2.

$$DPPH scavenging ability ( \% ) = \frac{Ac - As}{Ac} \times 100$$

where: A_{c}: absorbance of the DPPH solution, and Aₛ: absorbance of the samples.

### Example 2

To determine the effect of subcritical water on the conformational changes of protein, the intrinsic fluorescence intensity was measured, as shown in FIGS. 6A and 6B. Compared to the untreated sample, all subcritical water-hydrolyzed protein with pectin has appreciably lower fluorescence intensity. The decrease in the intensity could indicate the changes in the protein microenvironment which shift to a more polar setting. The unfolding of the protein has occurred, leading to the exposure of the interior hydrophobic clusters to the surface of the molecules.

The maximum emission wavelength is also an indicator of protein denaturation level (λmax). Compared to the untreated sample which had λmax at 382 nm, the subcritical water hydrolyzed proteins experience a slight red-shifting in λmax; in which, the λmax values range from 384 nm to 388 nm. This could be explained by the exposure of tryptophan residues to the aqueous environment. The interactions of aromatic amino acids with aqueous media could enhance the fluorescence quenching of water therefore resulting in the lower fluorescence intensity and the red shift of λmax.

### Example 3

The degree of hydrolysis (DH) is indicated by the amount of free amino groups in the hydrolysates. The higher DH demonstrates the protein is cleaved into free amino groups at higher extent. As shown in FIG. 1A, with increasing time, the DH increases and reaches the maximum value of 50.5% at 40 min, afterwards, the DH remains unchanged even with longer time of 50 and 60 min. The phenomenon is in part attributed to the dissociation constant (Kw) of water, which depends on temperature and pressure of the media. For that reason, the degree of hydrolysis does not further increase after the protein molecules are hydrolyzed by the available ions of H3O+ and OH-in the subcritical water medium. From 10 to 40 min, the DH is significantly enhanced due to better interaction between ionic species and the protein.

FIG. 1B shows the effect of temperature on the DH. From 160° C. to 200° C., the DH only increases slightly. However, the DH is significantly improved and peaked at 220° C. with the highest value of 64.8% (FIG. 1B). As it was mentioned earlier, the Kw value of water increases with temperature, Kw value at 220° C. is approximately 700 times higher than the one at ambient conditions. Consequently, the protein undergoes a more severe hydrolysis due to higher concentration of H3O+ and OH− and ions which could potentially break the peptide bonds in the protein; eventually increasing the amount of free amino groups in the hydrolysates. The samples with protein alone also showed the highest DH value at 220° C./40 min, however, compared to the hydrolysates of protein+pectin, they had significantly lower DH. Pectin, a polymer of D-galacturonic acids (GalA), can be hydrolyzed into its monomer due to the susceptibility of glycosidic bonds.

### Example 4

The changes in the molecular weight of peptide chains are monitored by size exclusion chromatography. FIG. 7 and FIG. 8 show the effect of reaction times. With longer time, the intensity of 4.3 kDa peak considerably increases. Also, the peak area of 4.3 kDa at 20 min and 40 min are 1.8 and 5.5 times higher than the one obtained at 10 min, respectively. This observation agrees with the degree of hydrolysis (FIG. 1A) where the highest DH is obtained at 40 min, which is likely due to the high content of peptides in the hydrolysate. Compared to reaction time, temperature had more pronounced effect in reducing the peptides molecular mass. With increasing temperature, the protein experiences better hydrolysis and the smaller peptides are therefore produced. The peptides found at 180° C. had molecular masses between 4.5 and 4.9 kDa with relatively low intensity from 53-55 mAu.

However, at higher temperature of 220° C., the peaks eluted at 30 and 31 min disappeared, instead, there is a prominent peak detected at 32 min with a considerably higher intensity, 153 mAu. The molecular mass of the identified peptides is 4.1 kDa. As seen in FIG. 7B, the intensity of peptides at 220° C. is approximately 3 times higher than the one obtained at 180° C. Furthermore, the area of 4.1 kDa peptides at 220° C. were 6.5 times higher than the one at 180° C. suggesting that the proteolytic activity of the media is enhanced at higher temperature.

### REFERENCES

Acquah, C., Zhang, Y., Dubé, M. A., & Udenigwe, C. C. (2020). Formation and characterization of protein-based films from yellow pea (Pisum sativum) protein isolate and concentrate for edible applications. Current Research in Food Science, 2, 61-69. https://doi.org/10.1016/J.CRFS.2019.11.008
Barbana, C., & Boye, J. I. (2010). Angiotensin I-converting enzyme inhibitory activity of chickpea and pea protein hydrolysates. Food Research International, 43 (6), 1642-1649. https://doi.org/10.1016/j.foodres.2010.05.003
Benjamin, M., Stéphane, R., Gérard, V., & Pierre-Yves, P. (2017). Pressurized water extraction of isoflavones by experimental design from soybean flour and Soybean Protein Isolate. Food Chemistry, 214, 9-15. https://doi.org/10.1016/j.foodchem.2016.07.053
Bogahawaththa, D., Bao Chau, N. H., Trivedi, J., Dissanayake, M., & Vasiljevic, T. (2019). Impact of selected process parameters on solubility and heat stability of pea protein isolate. LWT, 102, 246-253. https://doi.org/10.1016/J.LWT.2018.12.034
Boukid, F., Rosell, C. M., & Castellari, M. (2021). Pea protein ingredients: A mainstream ingredient to (re) formulate innovative foods and beverages. Trends in Food Science & Technology, 110, 729-742. https://doi.org/10.1016/J.TIFS.2021.02.040
Brunner, G. (2009). Near critical and supercritical water. Part I. Hydrolytic and hydrothermal processes. The Journal of Supercritical Fluids, 47 (3), 373-381. https://doi.org/10.1016/J.SUPFLU.2008.09.002
Chang, K., Jiang, W., & Liu, J. (2022). Effect of subcritical water treatment on the structure and foaming properties of egg white protein. Food Hydrocolloids, 124. https://doi.org/10.1016/j.foodhyd.2021.107241
Chao, D., & Aluko, R. E. (2018). Modification of the structural, emulsifying, and foaming properties of an isolated pea protein by thermal pretreatment. CYTA—Journal of Food, 16(1), 357-366. https://doi.org/10.1080/19476337.2017.1406536
Chao, D., He, R., Jung, S., & Aluko, R. E. (2013). Effect of pressure or temperature pretreatment of isolated pea protein on properties of the enzymatic hydrolysates. Food Research International, 54(2), 1528-1534. https://doi.org/10.1016/j.foodres.2013.09.020
Chao, D., Jung, S., & Aluko, R. E. (2018). Physicochemical and functional properties of high pressure-treated isolated pea protein. Innovative Food Science and Emerging Technologies, 45, 179-185. https://doi.org/10.1016/j.ifset.2017.10.014
Church, F. C., Swaisgood, H. E., Porter, D. H., & Catignani, G. L. (1983). Spectrophotometric Assay Using o-Phthaldialdehyde for Determination of Proteolysis in Milk and Isolated Milk Proteins. Journal of Dairy Science, 66(6), 1219-1227. https://doi.org/10.3168/jds.S0022-0302(83) 81926-2
Ziero, H.D., Ampese, L. C., Sganzerla, W. G., Torres-Mayanga, P. C., Timko, M. T., Mussatto, S. I., & Forster-Carneiro, T. (2022). Subcritical water hydrolysis of poultry feathers for amino acids production. The Journal of Supercritical Fluids, 181, 105492. https://doi.org/10.1016/J.SUPFLU.2021.105492
Ding, J., Liang, R., Yang, Y., Sun, N., & Lin, S. (2020). Optimization of pea protein hydrolysate preparation and purification of antioxidant peptides based on an in silico analytical approach. LWT, 123. https://doi.org/10.1016/j.lwt.2020.109126
Espinoza, A. D., & Morawicki, R. O. (2012). Effect of additives on subcritical water hydrolysis of whey protein isolate. Journal of Agricultural and Food Chemistry, 60(20), 5250-5256. https://doi.org/10.1021/jf300581r
Espinoza, A. D., Morawicki, R. O., & Hager, T. (2012). Hydrolysis of Whey Protein Isolate Using Subcritical Water. Journal of Food Science, 77(1), C20-C26. https://doi.org/10.1111/J.1750-3841.2011.02462.X
Garcia-Moscoso, J. L., Teymouri, A., & Kumar, S. (2015). Kinetics of Peptides and Arginine Production from Microalgae (Scenedesmus sp.) by Flash Hydrolysis. Industrial and Engineering Chemistry Research, 54(7), 2048-2058. https://doi.org/10.1021/IE5047279
Girgih, A. T., Chao, D., Lin, L., He, R., Jung, S., & Aluko, R. E. (2015). Enzymatic protein hydrolysates from high pressure-pretreated isolated pea proteins have better antioxidant properties than similar hydrolysates produced from heat pretreatment. Food Chemistry, 188, 510-516. https://doi.org/10.1016/j.foodchem.2015.05.024
Gomes, M. H. G., & Kurozawa, L. E. (2020). Improvement of the functional and antioxidant properties of rice protein by enzymatic hydrolysis for the microencapsulation of linseed oil. Journal of Food Engineering, 267. https://doi.org/10.1016/j.jfoodeng.2019.109761
Gorissen, S. H. M., Crombag, J. J. R., Senden, J. M. G., Waterval, W. A. H., Bierau, J., Verdijk, L. B., & van Loon, L. J. C. (2018). Protein content and amino acid composition of commercially available plant-based protein isolates. Amino Acids, 50(12), 1685-1695. https://doi.org/10.1007/s00726-018-2640-5
Hao, G., Cao, W., Li, T., Chen, J., Zhang, J., Weng, W., Osako, K., & Ren, H. (2019). Effect of temperature on chemical properties and antioxidant activities of abalone viscera subcritical water extract. Journal of Supercritical Fluids, 147, 17-23. https://doi.org/10.1016/j.supflu.2019.02.007
Ho, C. H. L., Cacace, J. E., & Mazza, G. (2007). Extraction of lignans, proteins and carbohydrates from flaxseed meal with pressurized low polarity water. LWT—Food Science and Technology, 40(9), 1637-1647. https://doi.org/10.1016/j.lwt.2006.12.003
Kang, K.-Y., & Chun, B.-S. (2004). Behavior of Hydrothermal Decomposition of Silk Fibroin to Amino Acids in Near-Critical Water. Korean J. Chem. Eng, 21(3), 654-659.
Klompong, V., Benjakul, S., Kantachote, D., & Shahidi, F. (2007). Antioxidative activity and functional properties of protein hydrolysate of yellow stripe trevally (Selaroides leptolepis) as influenced by the degree of hydrolysis and enzyme type. Food Chemistry, 102(4), 1317-1327. https://doi.org/10.1016/J.FOODCHEM.2006.07.016
Klost, M., & Drusch, S. (2019). Functionalisation of pea protein by tryptic hydrolysis-Characterisation of interfacial and functional properties. Food Hydrocolloids, 86, 134-140. https://doi.org/10.1016/j.foodhyd.2018.03.013
Klost, M., Giménez-Ribes, G., & Drusch, S. (2020). Enzymatic hydrolysis of pea protein: Interactions and protein fractions involved in fermentation induced gels and their influence on rheological properties. Food Hydrocolloids, 105, 105793. https://doi.org/10.1016/J.FOODHYD.2020.105793
Li, H., & Aluko, R. E. (2010). Identification and inhibitory properties of multifunctional peptides from pea protein hydrolysate. Journal of Agricultural and Food Chemistry, 58(21), 11471-11476. https://doi.org/10.1021/jf102538g
Li, H., Prairie, N., Udenigwe, C. C., Adebiyi, A. P., Tappia, P. S., Aukema, H. M., Jones, P. J. H., & Aluko, R. E. (2011). Blood Pressure Lowering Effect of a Pea Protein Hydrolysate in Hypertensive Rats and Humans. J. Agric. Food Chem, 59, 9854-9860. https://doi.org/10.1021/jf201911p
Lu, W., Chen, X. W., Wang, J. M., Yang, X. Q., & Qi, J. R. (2016). Enzyme-assisted subcritical water extraction and characterization of soy protein from heat-denatured meal. Journal of Food Engineering, 169, 250-258. https://doi.org/10.1016/j.jfoodeng.2015.09.006
Marcet, I., Álvarez, C., Paredes, B., & Diaz, M. (2016). The use of sub-critical water hydrolysis for the recovery of peptides and free amino acids from food processing wastes. Review of sources and main parameters. In Waste Management (Vol. 49, pp. 364-371). Elsevier Ltd. https://doi.org/10.1016/j.wasman.2016.01.009
Martinez-Monteagudo, S. I., & Saldaña, M. D. A. (2014). Chemical Reactions in Food Systems at High Hydrostatic Pressure. Food Engineering Reviews, 6(4), 105-127. https://doi.org/10.1007/S12393-014-9087-6/FIGURES/2
Mirzaei, M., Mirdamadi, S., Ehsani, M. R., Aminlari, M., & Hosseini, E. (2015). Purification and identification of antioxidant and ACE-inhibitory peptide from Saccharomyces cerevisiae protein hydrolysate. Journal of Functional Foods, 19, 259-268. https://doi.org/10.1016/J.JFF.2015.09.031
Ndlela, S. C., de Moura, J. M. L. N., Olson, N. K., & Johnson, L. A. (2012). Aqueous Extraction of Oil and Protein from Soybeans with Subcritical Water. Journal of the American Oil Chemists' Society, 89(6), 1145-1153. https://doi.org/10.1007/S11746-011-1993-7
Park, J. S., Jeong, Y. R., & Chun, B. S. (2019). Physiological activities and bioactive compound from layer (Pyropia yezoensis) hydrolysates by using subcritical water hydrolysis. The Journal of Supercritical Fluids, 148, 130-136. https://doi.org/10.1016/J.SUPFLU.2019.03.004
Pelgrom, P. J. M., Boom, R. M., & Schutyser, M. A. I. (2015). Method Development to Increase Protein Enrichment During Dry Fractionation of Starch-Rich Legumes. Food and Bioprocess Technology, 8(7), 1495-1502. https://doi.org/10.1007/S11947-015-1513-0/FIGURES/6
Phongthai, S., D'Amico, S., Schoenlechner, R., Homthawornchoo, W., & Rawdkuen, S. (2018). Fractionation and antioxidant properties of rice bran protein hydrolysates stimulated by in vitro gastrointestinal digestion. Food Chemistry, 240, 156-164. https://doi.org/10.1016/J.FOODCHEM.2017.07.080
Pinkowska, H., & Oliveros, E. (2014). Application of the doehlert matrix for the determination of the optimal conditions of hydrolysis of soybean protein in subcritical water. Industrial and Engineering Chemistry Research, 53(4), 1320-1326. https://doi.org/10.1021/ie403451b
Pourali, O., Asghari, F. S., & Yoshida, H. (2009). Sub-critical water treatment of rice bran to produce valuable materials. In Food Chemistry (Vol. 115, Issue 1, pp. 1-7). Elsevier. https://doi.org/10.1016/j.foodchem.2008.11.099
Powell, T., Bowra, S., & Cooper, H. J. (2016). Subcritical Water Processing of Proteins: An Alternative to Enzymatic Digestion? 21, 32. https://doi.org/10.1021/acs.analchem.6b01013
Pownall, T. L., Udenigwe, C. C., & Aluko, R. E. (2010). Amino Acid Composition and Antioxidant Properties of Pea Seed (Pisum sativum L.) Enzymatic Protein Hydrolysate Fractions. J. Agric. Food Chem, 58, 4712-4718. https://doi.org/10.1021/jf904456r
Qin, F., Johansen, A. Z., & Mussatto, S. I. (2018). Evaluation of different pretreatment strategies for protein extraction from brewer's spent grains. Industrial Crops and Products, 125, 443-453. https://doi.org/10.1016/J.INDCROP.2018.09.017
Ramachandraiah, K., Koh, B. B., Davaatseren, M., & Hong, G. P. (2017). Characterization of soy protein hydrolysates produced by varying subcritical water processing temperature. Innovative Food Science and Emerging Technologies, 43, 201-206. https://doi.org/10.1016/j.ifset.2017.08.011
Ramirez, C. S. V., Temelli, F., & Saldaña, M. D. A. (2021). Production of pea hull soluble fiber-derived oligosaccharides using subcritical water with carboxylic acids. The Journal of Supercritical Fluids, 178, 105349. https://doi.org/10.1016/J.SUPFLU.2021.105349
Reinkensmeier, A., Buβler, S., Schlüter, O., Rohn, S., & Rawel, H. M. (2015). Characterization of individual proteins in pea protein isolates and air classified samples. Food Research International, 76 (P1), 160-167. https://doi.org/10.1016/J.FOODRES.2015.05.009
Rogalinski, T., Herrmann, S., & Brunner, G. (2005). Production of amino acids from bovine serum albumin by continuous sub-critical water hydrolysis. The Journal of Supercritical Fluids, 36(1), 49-58. https://doi.org/10.1016/J.SUPFLU.2005.03.001
Rogalinski, T., Liu, K., Albrecht, T., & Brunner, G. (2008). Hydrolysis kinetics of biopolymers in subcritical water. The Journal of Supercritical Fluids, 46(3), 335-341. https://doi.org/10.1016/J.SUPFLU.2007.09.037
Sereewatthanawut, I., Prapintip, S., Watchiraruji, K., Goto, M., Sasaki, M., & Shotipruk, A. (2008). Extraction of protein and amino acids from deoiled rice bran by subcritical water hydrolysis. Bioresource Technology, 99(3), 555-561. https://doi.org/10.1016/j.biortech.2006.12.030
Simsek Kus, N. (2012). Organic reactions in subcritical and supercritical water. Tetrahedron, 68(4), 949-958. https://doi.org/10.1016/J.TET.2011.10.070
Sunphorka, S., Chavasiri, W., Oshima, Y., & Ngamprasertsith, S. (2012). Kinetic studies on rice bran protein hydrolysis in subcritical water. Journal of Supercritical Fluids, 65, 54-60. https://doi.org/10.1016/j.supflu.2012.02.017
Valdivieso Ramirez, C. S., Sanchez Gallego, J. E., Gänzle, M., Temelli, F., & Saldaña, M. D. A. (2021). Carboxylic acid-catalysed hydrolysis of polygalacturonic acid in subcritical water media. The Journal of Fluids, 169, 105103. Supercritical https://doi.org/10.1016/J.SUPFLU.2020.105103
Wang, M. P., Chen, X. W., Guo, J., Yang, J., Wang, J. M., & Yang, X. Q. (2019).
Stabilization of foam and emulsion by subcritical water-treated soy protein: Effect of aggregation state. Food Hydrocolloids, 87, 619-628. https://doi.org/10.1016/J.FOODHYD.2018.08.047
Watchararuji, K., Goto, M., Sasaki, M., & Shotipruk, A. (2008). Value-added subcritical water hydrolysate from rice bran and soybean meal. Bioresource Technology, 99(14), 6207-6213. https://doi.org/10.1016/j.biortech.2007.12.021
Waterborg, J. H. (2009). The Lowry Method for Protein Quantitation. 7-10. https://doi.org/10.1007/978-1-59745-198-7 2
Wen, C., Zhang, J., Yao, H., Zhou, J., Duan, Y., Zhang, H., & Ma, H. (2019). Advances in renewable plant-derived protein source: The structure, physicochemical properties affected by ultrasonication. Ultrasonics Sonochemistry, 53, 83-98. https://doi.org/10.1016/J.ULTSONCH.2018.12.036
Wiboonsirikul, J., Mori, M., Khuwijitjaru, P., & Adachi, S. (2013). Properties of Extract from Okara by Its Subcritical Water Treatment. Https://Doi.org/10.1080/10942912.2011.573119, 16(5), 974-982. https://doi.org/10.1080/10942912.2011.573119
Zhang, J., Wen, C., Zhang, H., Zandile, M., Luo, X., Duan, Y., & Ma, H. (2018). Structure of the zein protein as treated with subcritical water. International Journal of Food Properties, 21(1), 128-138. https://doi.org/10.1080/10942912.2017.1414839
Zhang, S. B., Wang, Z., & Xu, S. Y. (2008). Antioxidant and antithrombotic activities of rapeseed peptides. JAOCS, Journal of the American Oil Chemists' Society, 85(6), 521-527. https://doi.org/10.1007/s11746-008-1217-y
Zhao, Y., & Saldaña, M. D. A. (2019). Hydrolysis of cassava starch, chitosan and their mixtures in pressurized hot water media. The Journal of Supercritical Fluids, 147, 293-301. https://doi.org/10.1016/J.SUPFLU.2018.11.013

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A method of producing peptides from seed protein comprising:
mixing a seed protein concentrate with a catalyst to prepare a mixture;
dissolving the mixture in a buffer to prepare a suspension; and
hydrolysing the suspension in a subcritical water medium in a high pressure system that includes a temperature controller, a batch stirred reactor, and a heating mantle.

2. The method of claim 1 wherein hydrolysing the suspension comprises:
loading the suspension into the batch stirred reactor;
purging the reactor with a purge gas while stirring the suspension therein for a purge time;
pressurizing the reactor with a pressurizing gas to a reaction pressure;
heating the reactor to a reaction temperature;
maintaining the reaction pressure and the reaction temperature within the reactor for a reaction time;
cooling the reactor to a post-reaction temperature after the reaction time has elapsed and depressurizing the reactor; and
centrifuging a hydrolysate resulting from the hydrolysing.

3. The method of claim 1 wherein the catalyst is citrus pectin.

4. The method of claim 1 wherein the mixture is prepared by mixing the seed protein concentrate with the catalyst at a mass ration of 1:1 (w/w).

5. The method of claim 1 wherein the mixture is dissolved in the buffer at a pH above the isoelectric point of the protein.

6. The method of claim 1 wherein the buffer is a 0.2M phosphate buffer.

7. The method of claim 1 wherein the buffer has a pH 8.

8. The method of claim 1 further comprising stirring the suspension for a plurality of hours at a constant speed to hydrate the protein.

9. The method of claim 8 wherein the plurality of hours is at least eight hours.

10. The method of claim 1 wherein hydrolysing the suspension generates up to 65% degree of hydrolysis of the pea protein.

11. The method of claim 1 wherein the batch stirred reactor is a 600 mL reactor.

12. The method of claim 1 wherein the heating mantle is a 780 W heating mantle.

13. The method of claim 1 wherein at least one of the purge gas or the pressurizing gas is dinitrogen.

14. The method of claim 1 wherein the purge time is 12 minutes.

15. (canceled)

16. The method of claim 1 wherein the reaction pressure is 50 bar.

17. The method of claim 1 wherein the reaction temperature is 160° C. to 240° C.

18. The method of claim 1 wherein the reaction time is 10 to 60 minutes.

19. The method of claim 1 wherein the post-reaction temperature is 40° C.

20. The method of claim 1 wherein the hydrolysate is centrifuged at 8,000 g/20 min.

21. The method of claim 1 wherein the seed protein concentrate is pea protein concentrate.
